(19) United States
(12) Patent Application Publication
(10) Pub. No.: US 2008/0274487 A1
(43) Pub. Date: Nov. 6, 2008

(54) Use of Serum/Glucocorticoid-Regulated Kinase

(75) Inventors: Eckart Bartnik, Wiesbaden (DE); Thomas Aigner, Erlangen (DE); Uwe Dietz, Bad Soden (DE); Annette Brimmer, Frankfurt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 11/720,093

(22) PCT Filed: Dec. 1, 2005
(86) PCT No.: PCT/EP05/12802

§ 371 Date: Mar. 7, 2008

(30) Foreign Application Priority Data
Dec. 10, 2004 (DE) 10 2004 059 781.2

(51) Int. Cl. C12Q 1/48; G01N 33/68; C12Q 1/02

(52) U.S. Cl. 435/15; 436/86; 435/29

(57) ABSTRACT

The invention relates to the use of an SGK protein, a functional derivative or fragment thereof, or a nucleic acid coding for one such protein, fragment or derivative, in order to discover active ingredients for the prevention or treatment of degenerative cartilage changes.

The invention relates to the use of the serum/glucocorticoid-regulated kinase (SGK) for finding active ingredients. Further aspects of the invention relate to methods and test kits for identifying degenerative cartilage changes.

The kinase SGK-1 which is induced by serum and glucocorticoids was described for the first time in 1993 as immediate early gene in a rat mammary carcinoma cell line (Webster et al., 1993a; Webster et al., 1993b). There is in these cells, as early response to administration of glucocorticoids, an induction of SGK-1 gene activity and, as a consequence thereof, a significant increase in the amounts of SGK-1 mRNA. It was shown in further studies that SGK-1 and its inducibility occurs both in various cell lines and in cells of normal tissues (Brennan et al., 2000; Naray-Fejes-Toth et al., 2000; Cooper et al., 2001; Mikosz et al., 2001). SGK-1 belongs to a family of serine/threonine kinases of which to date three members are known and are referred to as SGK-1, SGK-2 and SGK-3. SGK-3 is also described under the names SGKL (SGK-like) and CISK. In its catalytic domain, the three isoforms are at least 80% homologous to one another at the protein level.

SGK-1 is expressed in virtually all tissues which have been tested to date, but the amounts of expressed mRNA vary widely depending on the nature of the tissue type investigated (Gonzalez-Robayna et al., 1999; Waldegger et al., 1999; Alliston et al., 2000; Klingel et al., 2000; Lang et al., 2000; Loffing et al., 2001; Fillon et al., 2002; Warntges et al., 2002a;). In addition, SGK-1 mRNA is found in typical embryonic tissues. During mouse embryogenesis, SGK-1 mRNA shows developed-dynamic changes in specific tissues of the embryo (decidua, yolk sack, otic vesicle) and is detectable during organogenesis in lung buds, brain, heart, liver, thymus etc. (Lee et al., 2001). SGK-2 is expressed with greatest abundance in epithelial tissues, for instance in the kidney, liver, pancreas and specific regions of the brain (Kobayashi et al., 1999b). SGK-3 has been detectable in all tissues tested to date and is to be found in particular in the adult heart and spleen (Kobayashi et al., 1999b; Liu et al., 2000).

However, no evidence of the expression of SGK in degenerating or degenerated cartilage tissue has emerged as yet.

Osteoarthrosis (OA) is one of the most commonly occurring degenerative joint disorders and leads, in the advanced stage, to loss of joint function. During the chronic course of the disease there is destruction of articular cartilage extending into the underlying bone tissue, making joint replacement operations necessary for patients affected. Besides the destruction of cartilage, pathological changes can also be observed in the synovial membrane and the ligaments. The disease is, like rheumatoid arthritis, accompanied in some cases by inflammatory processes, but differs from this arthritis.

The exact causes of the disorder are not as yet known, but various factors may be involved, such as metabolic changes, mechanical stress, genetic defects or joint injuries.

Irrespective of the original trigger, the common pathological feature of OA is degradation of articular cartilage. A main feature of the pathological condition of OA is proteolytic cleavage of collagens and proteoglycans. At the same time, a whole series of further processes occur, such as anabolic repair mechanisms, regenerative differentiation of cells or cell death. The detailed molecular mechanisms of these processes are still far from completely understood.

Healthy function of the adult cartilage derives from its unique biomechanical properties which ensure both resistance to high pressures and the necessary elasticity of the tissue. The specific organization of the cartilage tissue is decisive therefor. In contrast to most other tissues, cartilage cells are not in direct contact but are embedded separately from one another in an extracellular matrix (ECM). The macromolecules of this ECM guarantee the ability of the articular cartilage and the joints to function.

The basic structure of the ECM consists of a network formed by fibrils of collagens of type II, IX and XI. Proteoglycans are embedded therein, mainly aggrecan, resulting in an extremely high osmotic water-binding capacity. The swelling pressure generated thereby, in conjunction with the properties of the collagenous basic structure, guarantee the specific properties of cartilage.

A main feature of the pathogenesis of OA is the loss of the ECM of the cartilage and of the articular cartilage tissue. Functioning of the affected joint is thus restricted or lost. In addition, various symptomatic parameters appear during the course of the disease, such as, for example, pain. Current methods for treating osteoarthrosis are mostly confined to the alleviation of symptomatic complaints. Causal therapy leading to regression of the cartilage degeneration on the basis of medication is not possible according to the current state of knowledge. Particularly for this reason there is a considerable need for novel active ingredients for the prevention and/or treatment of osteoarthrosis. There is in addition a need for possibilities of early and reliable diagnosis of degenerative changes in articular cartilage in order to be able to initiate the treatment if possible in the initial stage of the disease. The object of the invention is accordingly to provide novel possibilities for identifying active ingredients against degenerative cartilage changes.

This object is achieved according to the invention by the use of an SGK protein, of a functional derivative or fragment thereof (also functional derivatives of such a fragment) or of a nucleic acid (and its functional, e.g. stabilized, derivatives) encoding such a protein, functional fragment or derivative for finding active ingredients for the prevention or treatment of degenerative cartilage changes.

The sequence of the human SGK genes is known. The coding polynucleotide sequences of these genes can be downloaded under the numbers NM_{—}005627, AF153609 (SGK-1), AF169034, AF186470 (SGK-2), NM_{—}013257, AF085233, AF169035 (SGK-3) from the NCBI nucleotide database. Likewise, the protein sequences are available from the NCBI protein database under the numbers NP_{—}005618 (SGK-1), NP_{—}733794, NP_{—}057360 (SGK-2), Q96BR1 (SGK-3). The human SGK genes are localized on different chromosomes. SGK-1 is located on chromosome 6 (6q23), SGK-2 on chromosome 20 (20q13.2), SGK-3 on chromosome 8 (8q12.3-8q13.1). NCBI is the National Center for Biotechnology Information (postal address: National Center for Biotechnology Information, National Library of Medicine, Building 38A, Bethesda, Md. 20894, USA; web address: www.ncbi.nhm.nih.gov). Cloning of the SGK-1 gene has been described inter alia in Webster et al., 1993a; Waldegger et al. 1997; 1998; cloning of SGK-2 and SGK-3 was described for the first time by Kobayashi et al., 1999b.

One feature distinguishing SGK from many other kinases is based on the stringent stimulus-dependent regulation of transcription, of cellular localization and of enzymatic activation (Firestone et al., 2003) of the molecule.

A large number of stimuli are known to activate the transcription of SGK-1. These include mineralocorticoids (Brennan et al., 2000; Shigaev et al., 2000; Bhargava et al., 2001), gonadotropins (Richards et al., 1995; Gonzalez-Robayna et al., 2000), 1,25(OH)₂D₃ (Akutsu et al., 2001), p 53, osmotic, cell-volume and hypotonic changes (Waldegger et al., 1997; Klingel et al., 2000; Waldegger et al., 2000; Rozansky et al., 2002; Warntges et al., 2002a), cytokines such as GM-CSF and TNF-alpha (Cooper et al., 2001) or by TGF-beta (Kumar et al., 1999; Waldegger et al., 1999; Lang et al., 2000). Induction of SGK takes place in further growth-dependent signaling pathways by serum (Webster et al., 1993a), insulin and IGF-1 (Kobayashi et al., 1999a; Park et al., 1999; Perrotti et al., 2001), FSH (Alliston et al., 1997), fibroblast- and platelet-derived growth factor (Davies et al., 2000), activators of the Erk signaling cascade (Hayashi et al., 2001) and TPA (Mizuno et al., 2001).

Some pathological changes involving SGK-1 activation are also known, such as in ischemic injuries to the brain (Imaizumi et al., 1994), viral hepatitis (Fillon et al., 2002), pulmonary fibrosis (Warntges et al., 2002b) or cardiac fibrosis (Funder 2001).

The implication of SGK in processes of degenerative joint disorders was, however, not previously known.

Extracellular stimuli do not in principle generally bring about an activation of SGK-1. Thus, for example, administration of heparin is followed by inhibition of SGK-1 transcription in proliferating smooth muscle cells of the vascular system (Delmolino et al., 1997).

In order to be converted into its functioning form, SGK-1 requires activation by phosphorylation. This is mediated by a signaling cascade in which phosphatidylinositol (PI)-3 kinase and the 3-phosphoinositide-dependent kinases PDK1 and PDK2 are involved.

Activation of SGK-1 by the PI-3 kinase signaling pathway is known to be a response to insulin, IGF and growth factors. Activation requires phosphorylation on two amino acid residues, threonine²⁵⁶ on the T loop and serine⁴²² on the hydrophobic motif of the protein. Phosphorylation on threonine²⁵⁶ is mediated by PDK1, and phosphorylation of serine⁴²² is said to be catalyzed by a putative PDK2 which is not as yet known (Kobayashi et al., 1999a; Park et al., 1999; Biondi et al., 2001).

Activation by PDK1 has been shown by means of mutations of the threonine²⁵⁶ and serine⁴²² phosphorylation sites. Mutation of threonine²⁵⁶ to alanine brings about an 80-90% reduction in phosphorylation and prevents PDK1-dependent activation of SGK-1. The same effect is observed when both threonine²⁵⁶ and serine⁴²² are mutated to alanine. By contrast, mutation of serine⁴²² to aspartate brings about an approx. 6-fold increase in the phosphorylation and activation by PDK1 (Kobayashi et al., 1999a).

The function of SGK in physiological or pathological processes is not as yet known. Concerning the function of SGK there is at present merely a series of investigations showing that SGK-1, -2 and -3 have a regulatory influence on channels of the cell membrane. Thus, it has been shown that the epithelial Na⁺ channel (ENaC), the principal transporter for mineralocorticoid-regulated Na⁺ reabsorption in the renal tubule, is a target molecule of SGK-1, SGK-2 and SGK-3 (Alvarez de la Rosa et al., 1999; Böhmer et al., 2000; Wagner et al., 2000a; Wang et al., 2001; Falefti et al., 2002; Friedrich et al., 2003). Interaction of SGK with ENaC takes place not by direct phosphorylation (Lang et al., 2000), but by inactivation of the ubiquitin ligase Nedd-4-2 (Debonneville et al., 2001; Snyder et al., 2002) as a result of phosphorylation by SGK. The amount and the residence time of ENaC in the cell membrane is increased thereby (Staub et al., 1997; Alvarez de la Rosa et al., 1999; Wagner et al., 2000a). In addition, it has been shown in a number of experiments that ROMK1 is a target molecule of SGK. However, ROMK1 is not directly regulated by SGK, but requires for this purpose the “Na⁺/H⁺ exchange regulating factor 2” (NHERF2) as mediator molecule (Shenolikar et al., 2001; Yun et al., 2003). The same applies to a further target molecule of SGK, the Na⁺/H⁺ transporter NHE3 (Yun et al., 2002). In addition, it has been shown in experiments on xenopus oocytes that SGK influences the Kv1.3 channel-dependent K⁺ current (Gamper et al., 2002b; Warntges et al., 2002a). It has additionally been reported that SGK regulates the amino acid transporters 42F/LAT and SN1 (Wagner et al., 2000b; Bohmer et al., 2003a,b).

However, despite these findings, it has to date been possible to reconstruct a picture of SGK function in physiological or pathological processes only in part.

Fragments of SGK are polypeptides or oligopeptides (or poly- or oligonucleotides encoding them) which differ through deletion of one or more amino acids from the ends or from the interior of the naturally occurring SGK, preferably human SGK and particularly preferably the SGK according to one of the sequences shown in SEQ ID No. 1, 2 or 3. Preferred examples of functional fragments comprise or consist of the SGK kinase domain (see Kobayashi et al., 1999a). Particularly preferred in this connection are the fragments which include sequences as shown in SEQ ID No. 7-12 and, in particular, consist thereof. Derivatives of SGK are polypeptides or oligopeptides having SGK function which differ through exchange of at least one amino acid and/or by addition of at least one amino acid and/or chemical modification of at least one amino acid, such as, for example, biotinylation, phosphorylation etc., from the naturally occurring SGK protein, preferably human SGK and in particular the SGK according to one of the sequences shown in SEQ ID No. 1, 2 or 3.

Functional fragments or derivatives of SGK are poly- or oligopeptides or poly- or oligonucleotides encoding them which have at least one function of SGK. Various functions of SGK are described hereinafter, e.g. regulation of the cell volume (Fillon et al., 2001), activation of epithelial Na channels (EnaC), (Debonneville et al., 2001); activation of Na⁺ transport (Rozansky et al., 2002 or Lang and Cohen, 2001) or of amino acid transport (Wagner et al., 2000), or regulation of the KCNE1-dependent K⁺ flux; the kinase activity of SGK, e.g. the phosphorylation of the ubiquitin ligase P or the phosphorylation of Nedd-4-2 at serine 444 and/or serine 338 (Synder et al., 2002); the capability of autophosphorylation or the ability to phosphorylate certain peptides (see, for example, Kobayashi et al., 1999a). The ability to interact with certain proteins, e.g. one of the aforementioned, likewise belongs to the functions of SGK. Particularly suitable in this connection is the ability of SGK to phosphorylate Ser and Thr residues in Arg-Xaa-Arg-Xaa-Xaa-Ser/Thr motifs (cf. Kobayashi et al., 1999a), especially in an oligo- or polypeptide which comprises the following sequence or consists thereof: GRPRTSSFAEG. The fragments or derivatives of a particular SGK subtype may moreover have at least one function which is common to all SGK subtypes (e.g. the ability of SGK to phosphorylate Ser and Thr residues in Arg-Xaa-Arg-Xaa-Xaa-Ser/Thr motifs or peptides of the sequence GRPRTSSFAEG). They preferably have a function which is specific for the respective subtype (e.g. different activity in the phosphorylation of particular synthetic peptides of different sequence, see, for example, Kobayashi et al., 1999a).

Test methods for finding protein activity are sufficiently well known to the relevant skilled worker. Thus, it is possible by conventional methods to determine for example the phosphorylation activity, the interaction with particular target proteins or fragments thereof, or the ability of SGK to modulate the activity of particular ion channels and transporters (concerning this, see also below). In an expedient embodiment, SGK is employed therefor as isolated molecule.

The term “isolated” in relation to polypeptides/proteins/polynucleotides/nucleic acids and their fragments and derivatives means in the context of the present invention that these molecules may have been either purified from natural sources or prepared recombinantly and purified (where the term “purified” also includes “partially purified”).

The preparation of isolated SGK molecules is sufficiently well known to the relevant skilled worker. It is thus possible on the basis of the known genomic or coding polynucleotide sequences for polynucleotides of desired length to be amplified by the polymerase chain reaction and then multiplied by cloning in host cells (concerning this, see inter alia the standard literature detailed below). The polymerase chain reaction (PCR) is an in vitro technique allowing polynucleotide segments flanked by two known sequences to be specifically amplified. It is sufficient in this connection if the sequence located 5′ from the sequence to be amplified is known. In this case, conventional techniques (e.g. restriction digestion, etc.) can be used to generate a fragment of the fragment to be amplified, to whose 3′ end a DNA molecule of known sequence is then ligated (so-called linker), so that the sequence to be amplified is finally flanked by two known sequences (so-called linker-mediated PCR, ImPCR).

Short, single-stranded DNA molecules (primers) which are complementary to the sequence regions of the DNA or RNA template which flank the polynucleotide to be amplified are used for the amplification. Under defined reaction conditions, the primers are extended, in the presence of deoxynucleotide triphosphates (dNTPs), along the single-stranded, denatured matrix by particular enzymes, called polymerases (DNA polymerases which recognize DNA as template and produce DNA strands, or reverse transcriptases which recognize RNA as template and produce DNA strands) to result in new DNA strands whose sequence is complementary to that of the template. It is ensured by choosing particular temperature sequences which are repeated periodically that the newly generated polynucleotide strands are denatured, primers then hybridize thereon and finally a new extension reaction takes place, etc. In order to be able to employ the temperatures necessary for denaturation without subsequent addition of new enzyme it is expedient to employ thermally stable polymerases, e.g. Taq polymerase (DNA polymerase from Thermus aquaticus). The selection of the suitable polymerase depends on the purpose of use and is known to the relevant skilled worker. Thus, when PCR is used for the purpose of cloning for example it is advantageous to choose polymerases with the ability to correct errors (so-called proofreading).

A typical PCR mixture comprises besides a polynucleotide template two suitable primers, for example in concentrations between 0.2 to 2 μm, also dNTPs, for example with concentrations of 200 μm per dNTP, also MgCl₂ with a concentration of 1-2 mM and 1-10 units of a thermally stable DNA polymerase such as, for example, Taq, and, for example, 0.01 to 20 μg of polynucleotide (i.e. DNA or RNA) template.

Primers or oligonucleotides are generated by chemical synthesis according to conventional protocols. The synthesis is moreover conventionally carried out by commercial suppliers such as MWG Biotech inter alia. cDNA of a wide variety of origins is commercially available, e.g. from suppliers such as Promega, Stratagene, etc. Suitable buffers and enzymes for carrying out PCR are likewise known to the relevant skilled worker and also commercially available. A further method for generating polynucleotide sequences in isolation consists of cloning the desired sequences by known methods, subsequent expression thereof in suitable organisms, preferably unicellular organisms such as, for example, suitable bacteria or yeast strains and subsequent (at least partial) purification of the expressed polynucleotide.

The PCR reaction for example is suitable for preparing a polynucleotide fragment for cloning. It is advantageous in this case to use at least one primer which has in its 5′ portion the recognition sequence of a restriction endonuclease (so-called restriction cleavage site).

It is advantageous for both primers to have such a cleavage site, it being possible for the two cleavage sites to be chosen to be identical or different. Conventional restriction enzymes are, for example: BamHI (GGATCC), ClaI (ATCGAT), EcoRI (GAATTC), EcoRV (GATATC), HindIII (MGCTT), NcoI (CCATGG), Sail (GTCGAC), XbaI (TCTAGA), etc. The selection of suitable restriction enzymes and suitable reaction conditions are sufficiently well known to the relevant skilled worker. For cloning, the DNA of a vector is cut (“digested”) by suitable restriction endonucleases which also have appropriate recognition sites in the primer sequences of the cDNA fragment to be cloned. The fragment generated for example by PCR is made compatible with the “digested” vector by isolation and treatment with the same restriction enzymes. Subsequently, after purification of vector and treated fragment, joining takes place by means of a DNA ligase under suitable reaction conditions (these are known to the skilled worker).

A vector is a circular or linear DNA molecule, for instance a plasmid, bacteriophage or cosmid, with whose aid DNA fragments can be specifically amplified in suitable organisms (so-called cloning). Suitable organisms are mostly unicellular organisms with high growth rates, such as, for example, bacteria or yeasts, but may also be cells from multicellular assemblages, such as, for example, cell lines generated from a wide variety of organisms (e.g. SF9 cells from Spodoptera Frugiperda, etc.). Suitable vectors are sufficiently well known to the skilled worker and are commercially available from biotechnology suppliers such as, for example, Roche Diagnostics, New England Biolabs, Promega, Stratagene, and many others.

The recombinant production of isolated polypeptides is also possible on the basis of the cloned polynucleotides by means of suitable specific vectors, which are likewise known in the prior art. This can take place by expression in suitable host cells, e.g. bacterial (preferably E. coli strains) or eukaryotic hosts (e.g. SF9 cells, etc.), the polynucleotides being in each case cloned into specific expression vectors and then inserted into the appropriate cells. Suitable methods for transformation and transfection are likewise known to the skilled worker (see, for example, appended standard literature), as are the conditions for cell culturing and induction of protein expression. Another possibility for the recombinant production of isolated polypeptides is in vitro translation. For this purpose too, the polynucleotide is cloned into a suitable vector and then expressed in vitro in suitable buffers and cell extracts (e.g. in reticulocyte lyzates). Vectors, further required reagents and conditions are likewise sufficiently well known and commercially available (e.g. from Invitrogen).

Three SGK subtypes (SGK 1 to 3) are known in the prior art. Accordingly, the use of at least one of these three family members, but preferably that of SGK 1, is preferred in the context of the various aspects of the present invention, e.g. the detection according to the invention of the expression of at least one of these three family members, in particular that of SGK 1.

The invention is based on the inventors' results surprisingly showing, by means of comparative gene expression analyses of samples of total cellular RNA from healthy and degenerated/degenerating cartilage tissue, that the serum/glucocorticoid-regulated kinase-1 (SGK-1) is expressed in degenerated/degenerating osteoarthrotic cartilage, whereas it is undetectable in healthy articular cartilage tissue. Detection of the molecule in cartilage tissue for the first time is involved here. In addition, further experiments by the inventors have provided the first evidence of the causal implication of SGK in the development of degenerative cartilage changes. The expression of SGK-1 in cartilage tissue moreover represents a completely new finding because no published investigations of the role of SGK in this tissue or of possible regulatory mechanisms of the expression of SGK under pathological and development-specific parameters in cartilage cells are known in the prior art. It is possible for the first time to conclude, on the basis of the inventors' studies, that SGK is specifically linked to pathological conditions of cartilage, for example in the framework of rheumatoid arthritis or of osteoarthrosis but especially within the framework of osteoarthrosis, and thus represents a key molecule for inducing cartilage-degrading processes.

Because of the great homology between the SGK family members, it can be assumed that this applies likewise to SGK-2 and SGK-3.

The identification of these relationships for the first time makes it possible to find active ingredients for the prevention or treatment of degenerative cartilage changes, by discovering by known test methods the effect of potential active ingredients on the activity of SGK and/or the level of SGK. The causal implication of SGK in the development of degenerative joint disorders moreover makes it possible to search specifically for therapeutic active ingredients which are targeted at regulatory mechanisms for restoring the normal cell physiology of the cartilage.

A further aspect of the invention accordingly relates to a method for identifying active ingredients for the prevention or treatment of degenerative cartilage changes with the steps
a. Contacting an SGK protein, a functional derivative or fragment thereof with a potential active ingredient.
b. Determining the SGK activity in the presence of the potential active ingredient.
c. Determining the SGK activity in the absence of the potential active ingredient.
d. Comparing the SGK activity from b with that from c), preferably determining whether the activity of SGK (protein, functional derivative or fragment thereof) is lower in the presence of the potential active ingredient than in its absence, and thus active ingredients are distinguished preferably on the basis of their ability to inhibit the activity of SGK.

A further aspect of the invention relates to a method for identifying active ingredients for the prevention or treatment of degenerative cartilage changes with the steps
a) Contacting a sample which comprises an SGK protein, a functional derivative or fragment of a nucleic acid coding therefor, with a potential active ingredient in a system which is capable at least of transcription.
b) Determining the amount of SGK protein, functional derivative or fragment thereof or of the mRNA encoding the latter, in the presence of the potential active ingredient.
c) Determining the amount of SGK protein, functional derivative or fragment thereof or of the mRNA encoding the latter, in the absence of the potential active ingredient.
d) Comparing the protein or mRNA amounts from b) and from c), preferably determining whether the amount of SGK (protein, functional derivative or fragment thereof or that of the mRNA encoding the latter) is lower in the presence of the potential active ingredient than in its absence, and thus active ingredients are distinguished preferably on the basis of their ability to reduce the amount of SGK available.

The invention further relates to a method for identifying active ingredients for the prevention or treatment of degenerative cartilage changes with the steps
a) Contacting a sample which comprises an SGK protein, a functional derivative or fragment thereof, or a nucleic acid coding therefor, with a potential active ingredient in a system which is capable at least of translation.
b) Determining the activity of the SGK protein, fragment or derivative in the presence of the potential active ingredient.
c) Determining the activity of the SGK protein, fragment or derivative in the absence of the potential active ingredient, preferably determining whether the SGK activity or amount (protein, functional derivative or fragment thereof or that of the mRNA encoding the latter) is lower in the presence of the potential active ingredient than in its absence, and thus active ingredients are distinguished preferably on the basis of their ability to inhibit the activity of SGK, or to reduce the amount of SGK available.

It is possible to establish whether the SGK activity is lower in the presence of the potential active ingredient than in its absence for example by determining the ability to phosphorylate synthetic peptides or other SGK substrates. (This also includes autophosphorylation). It is preferred in this connection to use poly- or oligopeptides which comprise the sequence motif Arg-Xaa-Arg-Xaa-Xaa-Ser/Thr, preferably the motif RPRTSS and in particular the motif GRPRTSSFAEG, or consist thereof. A further possibility is to determine the ability of SGK to modulate the activity of certain ion channels; this can be measured for example by means of intracellular ion concentrations or other methods known to the relevant skilled worker.

SGK activity refers in this connection to all functions of SGK (see also above). These may be modulated by the potential active ingredients at all levels affecting the protein activity (e.g. by direct interaction with the kinase domain; influencing post-translational modification, which may affect the protein activity, influencing protein folding or the activation by natural inhibitors or activators of SGK, such as, for example, PI3K). The amount of SGK relates to the balance of active SGK in a system, e.g. in a cell or a biochemical mixture at a particular time. The amount of SGK can moreover be modulated at all levels of SGK expression or SGK degradation (transcription including transcript processing and expulsion from the cell nucleus or translation or post-translational modification, which may affect protein stability and thus the balance of active SGK available).

Active ingredients having the ability to modulate, preferably reduce, the SGK activity and/or amount of SGK ought moreover, because of the functional implication of SGK in the change in cartilage cells to a degenerative status, inhibit, and preferably suppress, the development of degenerative cartilage changes and/or reduce, and preferably abolish, the effects of existing cartilage changes.

Suitable analytical methods or systems, so-called assays, which measure the activity or the concentration or amount of particular target molecules (here SGK) in the body (so-called targets, usually proteins or nucleic acids) as parameters of the activity of potential active ingredients are known in the state of the art. These may be for example in vitro assays, meaning biochemical assays with isolated or partly isolated components which are combined to give a reaction mixture by means of which the activity of potential active ingredients can be measured. Further possibilities therefor are cellular test systems (assays) in which the activity of the target protein (here SGK) and the activity of potential active ingredients on the activity of this target molecule in the cellular environment can be determined.

An assay in this connection is any type of analytical method by means of which it is possible to monitor a biological process. This conventionally entails molecular processes and signaling cascades which represent parts of physiological metabolic pathways and control mechanisms, but also pathological conditions, being mimicked in cellular or biochemical systems. The pharmacological activity of an active ingredient can then be determined on the basis of its ability to intervene in these pathways and mechanisms.

For use in the finding of active ingredients, especially the high-throughput screening for active ingredients, the assay must be reproducible and is preferably scalable and robust (meaning of low sensitivity to external influences). The assay should preferably be suitable for high-throughput screening of chemical substances for their ability to affect the activity of target molecules. The type of assay depends in this connection inter alia on the nature of the target molecule used (e.g. exact type or nature of biochemical basic molecule, e.g. polypeptide or polynucleotide) and the read-out, i.e. the parameters by means of which the activity of the target molecule is determined. Various assay types are known in the state of the art and in most cases are also commercially available from industrial suppliers.

Assays suitable for measuring the interaction of two binding partners comprise for example radioisotopic or fluorescent assays, e.g. fluorescence polarization assays as commercially available for example from Panvera, Perkin-Elmer Life Sciences (NEN™, LANCE™) or Packard BioScience (HTRF; ALPHAscreen™). The aforementioned assays are suitable for measuring the interaction of a labeled component with an unlabeled component (e.g. labeled proteins and their interaction with unlabeled ligands).

Further examples of assays comprise cellular assays in which a cell line stably (inducibly or constitutively, chromosomally or episomally) or transiently expresses ea recombinant protein as desired. These assays comprise for example reporter gene assays in which the regulation of a particular promoter or the regulation of a signal transduction pathway or of a member of a signal transduction cascade is measured by means of the activity of a reporter enzyme whose expression is under the control of the relevant promoter. For this type of assay it is necessary to generate a recombinant cell line which expresses the reporter gene under the control of a defined promoter which itself is subject to investigation, or which is regulated by the signal transduction cascade to be investigated. Suitable reporter enzymes are generally known to the relevant skilled worker and comprise firefly luciferase, renilla luciferase (both commercially available for example through Packard Reagents), β-galactosidase, etc. The selection of suitable cell lines is known to the relevant skilled worker and depends inter alia on the target of the assay or the read-out. These are ordinarily cell lines which are easy to culture and to transfect, such as, for example, HeLA, COS, CHO or NIH 3T3 cells.

Assays for measuring the intracellular ion concentration comprise for example so-called FLIPR™ (fluorometric imaging plate reader, commercially available from Molecular Devices) assays in which an argon laser light source in combination with a cooled CCD camera allows simultaneous measurement of the intracellular ion concentration (e.g. Na⁺ or Ca²⁺, etc.) of different mixtures in cells (e.g. cells which recombinantly or naturally express particular ion channels) which are cultured on 384-well plates. It is possible by means of these FLIPR™ assays to measure for example the intracellular calcium levels by means of particular fluorochromes, e.g. Fluo-3, Fluo-4, the intracellular pH by using BCECF, BCPCF or specific FLIPR™ assay kits, changes in the membrane potential by using for example DiBAC or specific FLIPR™ assay kits or changes in the membrane polarization. Other dyes are suitable for determining the concentration of other ions such as zinc, sodium, etc. and are likewise known in the state of the art and commercially available.

Examples of assays and dyes suitable for determining the activity of particular ion channels (which, for example, control intracellular ion concentrations and whose activity can thus be determined on the basis of the intracellular concentration of particular ions) are those which respond sensitively to changes in the membrane potential, e.g. DiBAC or the membrane potential assay kit for the FLIPR™ assay from Molecular Devices, measurement of the mitochondrial membrane polarization by JC-1 dye with the FLIPR™ technology; ion-sensitive dyes such as Fluo-3, Fluo-4 or the calcium assay kit from Molecular Devices with the FLIPR™ technology for determining intracellular ion concentrations; sodium-sensitive dyes, e.g. from Molecular Probes, for determining the intracellular sodium concentration; for determining the intracellular calcium concentration for example assays based on patch clamping or on rubidium ion efflux measurement by means of atomic adsorption spectroscopy, etc. Further automatic devices and robots for measuring and determining particular changes and states in cells are known to the relevant skilled worker and comprise for example the Acumen™ detector from Acumen Bioscience, a fluorescence-based laser scanning reading device which makes the three-dimensional reconstitution of the distribution of suitably labeled objects possible.

Examples suitable for measuring the protein phosphorylation or kinase activity are fluorescence polarization, e.g. commercially available through Panvera, homogeneous time resolved fluorescence (HTRF, C is Bio) or LANCE™ assays (Perkin Elmer Life Sciences) or the amplified luminescent proximity homogeneous assay (ALPHAScreen™ from Packard BioScience).

Other types of assays and other types of read-outs are likewise sufficiently well known to the relevant skilled worker.

A further aspect of the invention relates to a high-throughput screen for finding active ingredients based on one of the aforementioned methods. High-throughput screen means in the context of this invention that the method is carried out on very small scales, for example on 96-, 386- or 1536-well plates or in mixtures with small volumes in the region of a few milliliters down to a few nanoliters, so that a large number of mixtures, e.g. 500 000 or more, can be analyzed within a very short time.

The aforementioned uses and methods of the invention, and the aforementioned high-throughput screen serve to find active ingredients for the treatment and/or prevention of degenerative cartilage changes. They serve in particular to find active ingredients which are able to inhibit the ability of SGK to elicit and expedite such degenerative processes. In the context of the various aspects of this invention, inhibitory means suitable for inhibiting the function of at least one of the SGK family members, preferably at least that of SGK1. This inhibition may derive from a wide variety of mechanisms, for example by lowering the cellular SGK level (e.g. by reducing the transcription and/or translation, lowering the stability of protein or mRNA, etc.), inhibiting the enzymatic activity of SGK or negatively influencing upstream or downstream signaling pathways. Lowering the cellular SGK level or inhibiting the enzymatic activity is preferred in this connection.

In addition, the invention relates to a method for identifying developing and existing degenerative cartilage changes which comprises the detection of SGK in cartilage tissue.

The SGK detection takes place in this connection by methods known to the skilled worker. Thus, it is possible by suitable methods such as, for instance, detection of SGK in the degenerated or degenerating cartilage on the basis of immunohistochemical detections of SGK protein or RNA (for example immunohistochemical staining of histological sections of tissue samples, immunoblots, Elisas or protein microarrays) or by detecting SGK RNA by means of Northern blotting, quantitative PCR, in situ hybridizations or DNA microarrays, etc., to use the presence of SGK in the cartilage tissue for identifying an existing or developing cartilage degeneration. Further suitable detection methods for SGK expression are known to the relevant skilled worker. Suitable techniques for taking tissue samples, e.g. cartilage or synovial fluid containing abraded cartilage cells, and their processing for the various analytical methods are likewise sufficiently well known to the skilled worker.

Suitable antibodies for detecting the SGK family or single SGK family members are commercially available (Santa Cruz Biotechnology, Europa Bioproducts Ltd), or can be produced in rabbits by using SGK peptides, e.g. polypeptides having the sequences shown in SEQ ID No. 4, 5, 6 or SEQ ID No. 7-12, by standard protocols (see, for example, the standard literature indicated hereinafter). Their production is conventionally undertaken commercially by various suppliers, e.g. by BioTrend, Cologne, Germany. Antisera or antibodies which are specific for one of the SGK family members are produced in this connection preferably by using polypeptide fragments of the C-terminal noncatalytic region of the respective family member, e.g. of the 100, preferably 80 or fewer C-terminal amino acids or particularly preferably polypeptide fragments selected from the 100, preferably 85 N-terminal amino acids of the SGK proteins, in particular fragments consisting of 20 to 100, 50 to 90 and particularly preferably of the 85 N-terminal amino acids of the respective SGK family member (in the case of SGK2 starting from the sequence of SGK2β; particularly suitable for SGK2α for this purpose are the 21 N-terminal amino acids; cf. also Kobayashi et al., 1999b).

The selection and preparation of suitable probes for hybridizing immobilized nucleic acids on Northern and Southern blots, DNA microarrays, or tissue sections in situ, and of oligonucleotide primers for PCR is sufficiently well known to the relevant skilled worker (concerning this, see also the standard literature indicated hereinafter). Suitable for differential detection in this connection are in particular probes which code for the abovementioned, low-homology regions of the various SGK family members, e.g. probes selected from the first 255 nucleotides of the coding sequence (corresponding to the 85 low-homology N-terminal amino acids of the various SGK family members; and for SGK2α probes selected from the first 63 nucleotides of the coding sequence corresponding to the low-homology N-terminal 21 amino acids of the SGK2α protein; see above). Suitable in this connection for detecting SGK1 expression are preferably probes having the sequence shown in SEQ ID No. 1 (FIG. 1), SEQ ID No. 7 (FIG. 7) or SEQ ID No. 13 (FIG. 8).

In a preferred embodiment, SGK1 is detected in the method of the invention.

The method of the invention for identifying degenerative cartilage changes preferably comprises the following steps:
a) Measuring the content of SGK protein or mRNA of an isolated cartilage sample to be investigated;
b) Comparing with the content of SGK protein or mRNA of an isolated sample of nondegenerated cartilage,
   where the amount of SGK in degenerated or degenerating cartilage is larger than that in nondegenerated cartilage.

These methods of the invention allow developing or existing degenerative cartilage changes to be diagnosed simply and reliably by appropriate analysis of removed synovial fluid or tissue samples.

The invention further relates to a test kit for diagnosing degenerative cartilage changes comprising at least one means for detecting SGK. A kit of parts (or “kit” for short) in the context of the present invention means the combination of said components which have been combined into a functional unit in spatial juxtaposition to each other and which may where appropriate comprise further components.

The means is preferably an antibody against at least one SGK protein or protein fragment and/or a primer set and/or a nucleic acid probe (the two latter for detecting mRNA expression of at least one SGK mRNA). The preparation of such means is sufficiently well known to the relevant skilled worker. The kit may additionally comprise further advantageous components such as suitable buffers, enzymes, instructions for use, etc. Such components and necessary packaging means are known to the skilled worker from commercially available diagnostic test kits.

In a preferred embodiment of the various subject matters and aspects of the invention, SGK is a polynucleotide, preferably human SGK or a fragment or derivative thereof and comprises or consists particularly preferably of one of the following sequences:
a) SEQ ID No. 1, SEQ ID No. 2 or SEQ ID No. 3
b) A sequence which hybridizes with at least one of the sequences according to a) under stringent conditions and codes for a polypeptide having SGK function.
c) A sequence which, on the basis of the genetic code, is degenerate in relation to one of the sequences according to a or b and codes for a polypeptide having SGK function.
d) A fragment of one of the sequences according to a), b) or c) which codes for a polypeptide having SGK function, preferably a fragment having one of the sequences shown in SEQ ID No. 7-9 (FIG. 7).
e) A sequence generated starting from one of the preceding sequences a), b), c) or d) by exchange of one or more bases, where the base exchange does not, or does not exclusively, take place on the basis of the degeneracy of the genetic code, and which codes for a polypeptide having SGK function.

In a further preferred embodiment, the SGK polynucleotide comprises or consists of a nucleotide sequence shown in SEQ ID No. 1, SEQ ID No. 2 or SEQ ID No. 3.

The SGK fragments used according to the invention comprise or consist preferably of one of the nucleotide sequences shown in SEQ ID No. 7, SEQ ID No. 8, or SEQ ID No. 9 (polynucleotide fragments shown in FIG. 7).

In another preferred embodiment of the various subject matters and aspects of the invention, SGK is a polypeptide, preferably human SGK or a fragment or derivative thereof and is particularly preferably encoded by a nucleic acid which has at least one of the following sequences or consists thereof:
f) SEQ ID No. 1, SEQ ID No. 2 or SEQ ID No. 3
g) A sequence which hybridizes with at least one of the sequences according to a) under stringent conditions and codes for a polypeptide having SGK function.
h) A sequence which, on the basis of the genetic code, is degenerate in relation to one of the sequences according to a or b and codes for a polypeptide having SGK function.
i) A fragment of one of the sequences according to a), b) or c) which codes for a polypeptide having SGK function, preferably a fragment having one of the sequences shown in SEQ ID No. 7-9 (FIG. 7).
j) A sequence generated starting from one of the preceding sequences a), b), c) or d) by exchange of one or more bases, where the base exchange does not, or does not exclusively, take place on the basis of the degeneracy of the genetic code, and which codes for a polypeptide having SGK function.

In a further preferred embodiment, the SGK polypeptide comprises or consists of an amino acid sequence shown in SEQ ID No. 4, SEQ ID No. 5 or SEQ ID No. 6.

The SGK fragments used according to the invention comprise or consist preferably of one of the amino acid sequences shown in SEQ ID No. 10, SEQ ID No. 11, or SEQ ID No. 12 (FIG. 7).

Stringency describes reaction conditions which influence the specificity of the hybridization or annealing of two single-stranded nucleic acid molecules to one another. The stringency and thus also the specificity of a reaction depends in this connection inter alia on the temperature and the buffer conditions: thus, the stringency and therefore also the specificity can be increased by raising the temperature and lowering the ionic strength. Low-stringency conditions (i.e. also lower reaction or hybridization specificity) exist for example when the hybridization is carried out at room temperature in 2×SCC solution. High-stringency conditions by contrast apply for example when hybridization is at 68° C. in 0.1×SSC and 0.1% SDS solution.

Hybridization under stringent conditions in the sense of this application preferably means moreover
1) Hybridization of the labeled probe with the sample to be investigated at 60 to 70° C. and preferably 65° C. or in the case of oligonucleotides 5° C. below the melting point of the double strand of oligonucleotide and sample (this melting point is also called the “annealing temperature”) overnight in 50 mM Tris pH 7.5, 1 M NaCl, 1% SDS, 10% dextran sulfate, 0.5 mg/ml denatured herring or salmon sperm DNA.
2) Washing in 2×SSC at room temperature for 10 minutes.
3) Washing in 1×SSC/0.1% SDS at 60 to 70° C. and preferably 65° C. (or in the case of oligonucleotides 5° C. below the annealing temperature) for 30 minutes.
4) Washing in 0.2×SSC/0.1% SDS at 60 to 70° C. and preferably 65° C. (or in the case of oligonucleotides 5° C. below the annealing temperature) for 30 minutes.
5) Washing in 0.1×SSC/0.1% SDS at 60 to 70° C. and preferably 65° C. (or in the case of oligonucleotides 5° C. below the annealing temperature) for 30 minutes.

Oligonucleotides are preferably DNA fragments having a length of between 15 to 30, preferably 20 nucleotides. The annealing temperature is in this case determined by the formula Tm=2×(number of A+T)+4×(number of G+C)° C. A 2×SSC or 0.1×SSC solution is prepared for example by appropriate dilution of a 20×SSC solution. The 20×SSC solution consists of: 3M NaCl/0.3 M Na citrate×2H₂O.

Before the hybridization is carried out, the polynucleotides to be investigated, where appropriate after electrophoretic fractionation (so-called Southern (DNA) or Northern blot (RNA)), or without electrophoretic fractionation (so-called dot or slot blot), are transferred to a suitable membrane, e.g. a nylon or nitrocellulose membrane. The hybridization takes place with a probe labeled in a suitable way. Thus, radiolabeling or labeling with fluorescent dyes are expedient, but other types of labeling are likewise conceivable. The probe is a single-stranded polyribo- or polydeoxyribonucleotide which is per se in single-stranded form or is usually double-stranded, but is employed in the denatured state. This probe binds by base pairing to the DNA or RNA probe which is in turn in single-stranded form.

The various amino acid and nucleotide sequences are to be found in the figures.

The various subject matters and aspects of the invention are particularly suitable for use in cases of arthritic disorders, preferably osteoarthrosis or rheumatoid arthritis, and in particular osteoarthrosis.

### REFERENCES

Akutsu, N., Lin, R., Bastien, Y., Bestawros, A., Enepekides, D. J., Black, M. J., and White, J. H. (2001). Regulation of gene Expression by 1 alpha,25-dihydroxyvitamin D3 and Its analog EB1089 under growth-inhibitory conditions in squamous carcinoma Cells. Mol. Endocrinol., 15, 1127-1139.
Ali, S., Wei, Y., Lerea, K. M., Becker, L., Rubin, C. S., and Wang, W. (2001). PKA-induced stimulation of ROMK1 channel activity is governed by both tethering and non-tethering domains of an A kinase anchor protein. Celi Physiol Biochem., 11, 135-142.
Alliston, T. N., Gonzalez-Robayna, I. J., Buse, P., Firestone, G. L., and Richards, J. S. (2000). Expression and localization of serum/glucocorticoid-induced kinase in the rat ovary: relation to follicular growth and differentiation. Endocrinology, 141, 385-395.
Alliston, T. N., Maiyar, A. C., Buse, P., Firestone, G. L., and Richards, J. S. (1997). Follicle stimulating hormone-regulated expression of serum/glucocorticoid-inducible kinase in rat ovarian granulosa cells: a functional role for the Sp1 family in promoter activity. Mol. Endocrinol., 11, 1934-1949.
Alvarez, d. I. R., Zhang, P., Naray-Fejes-Toth, A., Fejes-Toth, G., and Canessa, C. M. (1999). The serum and glucocorticoid kinase sgk increases the abundance of epitheiial sodium channels in the plasma membrane of Xenopus oocytes. J Biol Chem, 274, 37834-37839.
Bhargava, A., Fullerton, M. J., Myles, K., Purdy, T. M., Funder, J. W., Pearce, D., and Cole, T. J. (2001). The serum- and glucocorticoid-induced kinase is a physiological mediator of aldosterone action. Endocrinology, 142, 1587-1594.
Biondi, R. M., Kieloch, A., Currie, R. A., Deak, M., and Alessi, D. R. (2001). The PIF-binding pocket in PDK1 is essential for activation of S6K and SGK, but not PKB. EMBO J, 20, 4380-4390.
Boehmer, C., Okur, F., Setiawan, I., Broer, S., and Lang, F. (2003a). Properties and regulation of glutamine transporter SN1 by protein kinases SGK and PKB. Biochem. Biophys. Res. Commun., 306, 156-162.
Boehmer, C., Wilhelm, V., Palmada, M., Wallisch, S., Henke, G., Brinkmeier, H., Cohen, P., Pieske, B., and Lang, F. (2003b). Serum and glucocorticoid inducible kinases in the regulation of the cardiac sodium channel SCN5A. Cardiovasc Res., 57, 1079-1084.
Boehmer, C., Wagner, C. A., Beck, S., Moschen, I., Melzig, J., Werner, A., Lin, J. T., Lang, F., and Wehner, F. (2000). The shrinkage-activated Na(+) conductance of rat hepatocytes and its possible correlation to rENaC. Cell Physiol Biochem., 10, 187-194.
Brennan, F. E. and Fuller, P. J. (2000). Rapid upregulation of serum and glucocorticoid-regulated kinase (sgk) gene expression by corticosteroids in vivo. Mol Cell Endocrinol., 166, 129-136.
Chen, S. Y., Bhargava, A., Mastroberardino, L., Meijer, O. C., Wang, J., Buse, P., Firestone, G. L., Verrey, F., and Pearce, D. (1999). Epithelial sodium channel regulated by aldosterone-induced protein sgk. Proc. Natl. Acad. Sci. U.S.A, 96, 2514-2519.
Cooper, M. S., Bujalska, I., Rabbitt, E., Walker, E. A., Bland, R., Sheppard, M. C., Hewison, M., and Stewart, P. M. (2001). Modulation of 11beta-hydroxysteroid dehydrogenase isozymes by proinflammatory cytokines in osteoblasts: an autocrine switch from glucocorticoid inactivation to activation. J Bone Miner. Res., 16, 1037-1044.
Davies, S. P., Reddy, H., Caivano, M., and Cohen, P. (2000). Specificity and mechanism of action of some commonly used protein kinase inhibitors. Biochem. J, 351, 95-105.
Debonneville, C., Flores, S. Y., Kamynina, E., Plant, P. J., Tauxe, C., Thomas, M. A., Munster, C., Chraibi, A., Pratt, J. H., Horisberger, J. D., Pearce, D., Loffing, J., and Staub, O. (2001). Phosphorylation of Nedd-4-2 by Sgkl regulates epithelial Na(+) channel cell surface expression. EMBO J, 20, 7052-7059.
Delmolino, L. M. and Castellot, J. J., Jr. (1997). Heparin suppresses sgk, an early response gene in proliferating vascuiar smooth muscle cells. J Cell Physiol, 173, 371-379.
Djelidi, S., Beggah, A., Courtois-Coutry, N., Fay, M., Cluzeaud, F., Viengchareun, S., Bonvalet, J. P., Farman, N., and Blot-Chabaud, M. (2001). Basolateral translocation by vasopressin of the aldosterone-induced pool of latent Na-K-ATPases is accompanied by alpha1 subunit dephosphorylation: study in a new aldosterone-sensitive rat cortical collecting duct cell line. J. Am. Soc. Nephrol., 12, 1805-1818.
Eisen, M. B. Spellman, P. T., Brown. P. O., Botstein, D. (1998). Cluster analysis and display of genome-wide expression patterns. Proc. Natl. Acad. Sci. USA 95(25):14863-14868.
Embark, H. M., Bohmer, C., Vallon, V., Luft, F., and Lang, F. (2003). Regulation of KCNE1-dependent K(+) current by the serum and glucocorticoid-inducible kinase (SGK) isoforms. Pflugers Arch, 445, 601-606.
Faletti, C. J., Perrotti, N., Taylor, S. I., and Blazer-Yost, B. L. (2002). sgk: an essential convergence point for peptide and steroid hormone regulation of ENaC-mediated Na⁺ transport. Am. J. Physiol Cell Physiol, 282, C494-C500.
Fillon, S., Warntges, S., Matskevitch, J., Moschen, I., Setiawan, I., Gamper, N., Feng, Y. X., Stegen, C., Friedrich, B., Waldegger, S., Broer, S., Wagner, C. A., Huber, S. M., Klingel, K., Vereninov, A., Lang, F. (2001). Serum- and glucocorticoid-dependent kinase, cell volume, and the regulation of epithelial transport. Comp. Biochem. Physiol. A Mol. Integr. Physiol. 130(3):367-376.
Fillon, S., Klingel, K., Warntges, S., Sauter, M., Gabrysch, S., Pestel, S., Tanneur, V., Waldegger, S., Zipfel, A., Viebahn, R., Haussinger, D., Broer, S., Kandolf, R., and Lang, F. (2002). Expression of the serine/threonine kinase hSGK1 in chronic viral hepatitis. Cell Physiol Biochem., 12, 47-54.
   Ref ID: 1
Firestone, G. L., Giampaolo, J. R., and O'Keeffe, B. A. (2003). Stimulus-dependent regulation of serum and glucocorticoid inducible protein kinase (SGK) transcription, subcellular localization and enzymatic activity. Cell Physiol Biochem., 13, 1-12.
Friedrich, B., Feng, Y., Cohen, P., Risler, T., Vandewalle, A., Broer, S., Wang, J., Pearce, D., Lang, F. (2003). The serine/threonine kinases SGK2 and SGK3 are potent stimulators of the epithelial Na(+) channel alpha, beta, gamma-EnaC. Pflugers Arch. 445(6):693-696.
Funder, J. (2001). Mineralocorticoids and cardiac fibrosis: the decade in review. Clin Exp Pharmacol Physiol, 28, 1002-1006.
Gamper, N., Fillon, S., Feng, Y., Friedrich, B., Lang, P. A., Henke, G., Huber, S. M., Kobayashi, T., Cohen, P., and Lang, F. (2002a). K+ channel activation by all three isoforms of serum- and glucocorticoid-dependent protein kinase SGK. Pflugers Arch, 445, 60-66.
Gamper, N., Fillon, S., Huber, S. M., Feng, Y., Kobayashi, T., Cohen, P., and Lang, F. (2002b). IGF-1 up-regulates K+ channels via PI3-kinase, PDK1 and SGK1. Pflugers Arch, 443, 625-634.
Giebisch, G. (1998). Renal potassium transport: mechanisms and regulation. Am. J. Physiol, 274, F817-F833.
Gonzalez-Robayna, I. J., Alliston, T. N., Buse, P., Firestone, G. L., and Richards, J. S. (1999). Functional and subcellular changes in the A-kinase-signaling pathway: relation to aromatase and Sgk expression during the transition of granulosa cells to luteal cells. Mol. Endocrinol., 13, 1318-1337.
Gonzalez-Robayna, I. J., Falender, A. E., Ochsner, S., Firestone, G. L., and Richards, J. S. (2000). Follicle-Stimulating hormone (FSH) stimulates phosphorylation and activation of protein kinase B (PKB/Akt) and serum and glucocorticoid-Induced kinase (Sgk): evidence for A kinase-independent signaling by FSH in granulosa cells. Mol. Endocrinol., 14, 1283-1300.
Hayashi, M., Tapping, R. I., Chao, T. H., Lo, J. F., King, C. C., Yang, Y., Lee, J. D. (2001). BMK1 mediates growth factor-induced cell proliferation through direct cellular activation of serum and glucocorticoid-inducible kinase. J. Biol. Chem. 276(12):8631-8634.
Imaizumi, K., Tsuda, M., Wanaka, A., Tohyama, M., and Takagi, T. (1994). Differential expression of sgk mRNA, a member of the Ser/Thr protein kinase gene family, in rat brain after CNS injury. Brain Res Mol Brain Res, 26, 189-196.
Klingel, K., Warntges, S., Bock, J., Wagner, C. A., Sauter, M., Waldegger, S., Kandolf, R., and Lang, F. (2000). Expression of cell volume-regulated kinase h-sgk in pancreatic tissue. Am. J. Physiol Gastrointest. Liver Physiol, 279, G998-G1002.
Kobayashi, T. and Cohen, P. (1999a). Activation of serum- and glucocorticoid-regulated protein kinase by agonists that activate phosphatidylinositide 3-kinase is mediated by 3-phosphoinositide-dependent protein kinase-1 (PDK1) and PDK2. Biochem. J, 339 (Pt 2), 319-328.
Kobayashi, T., Deak, M., Morrice, N., and Cohen, P. (1999b). Characterization of the structure and regulation of two novel isoforms of serum- and glucocorticoid-induced protein kinase. Biochem. J, 344 Pt 1, 189-197.
Kumar, J. M., Brooks, D. P., Olson, B. A., and Laping, N. J. (1999). Sgk, a putative serine/threonine kinase, is differentially expressed in the kidney of diabetic mice and humans. J. Am. Soc. Nephrol., 10, 2488-2494.
Lang, F., Cohen, P. (2001). The regulation and physiological roles of serum and glucocorticoid-induced protein kinase. Sci. STKE (108):RE17
Lang, F., Klingel, K., Wagner, C. A., Stegen, C., Warntges, S., Friedrich, B., Lanzendorfer, M., Melzig, J., Moschen, I., Steuer, S., Waldegger, S., Sauter, M., Paulmichl, M., Gerke, V., Risler, T., Gamba, G., Capasso, G., Kandolf, R., Hebert, S. C., Massry, S. G., and Broer, S. (2000). Deranged transcriptional regulation of cell-volume-sensitive kinase hSGK in diabetic nephropathy. Proc. Natl. Acad. Sci. U.S.A, 97, 8157-8162.
Lee, E., Lein, E. S., Firestone, G. L. (2001). Tissue-specific expression of the transcriptionally regulated serum and glucocorticoid-inducible preotein kinase (sgk) during embryogenesis. Mech. Dev. 103(1-2):177-81
Liu, D., Yang, X., and Songyang, Z. (2000). Identification of CISK, a new member of the SGK kinase family that promotes IL-3-dependent survival. Curr. Biol, 10, 1233-1236.
Loffing, J., Zecevic, M., Feraille, E., Kaissling, B., Asher, C., Rossier, B. C., Firestone, G. L., Pearce, D., and Verrey, F. (2001). Aldosterone induces rapid apical translocation of ENaC in early portion of renal collecting system: possible role of SGK. Am. J. Physiol Renal Physiol, 280, F675-F682
Maier et al., 1997; in: Automation for genome characterisation. Ed T. J. Beuelsdijk.; Publishers: J. Wiley, New York
Meier-Ewert, S. et al., Sebastian. Maier Elmar. Ahmadi Ali. Curtis Jon. Lehrach Hans. An automated approach to generating expressed sequence catalogues. Nature (London). 361(6410). 1993. 375-376
Mikosz, C. A., Brickley, D. R., Sharkey, M. S., Moran, T. W., and Conzen, S. D. (2001). Glucocorticoid receptor-mediated protection from apoptosis is associated with induction of the serine/threonine survival kinase gene, sgk-1. J Biol Chem, 276, 16649-16654.
Mizuno, H. and Nishida, E. (2001). The ERK MAP kinase pathway mediates induction of SGK (serum- and glucocorticoid-inducible kinase) by growth factors. Genes Cells, 6, 261-268.
Naray-Fejes-Toth, A., Canessa, C., Cleaveland, E. S., Aidrich, G., and Fejes-Toth, G. (1999). sgk is an aldosterone-induced kinase in the renal collecting duct. Effects on epithelial na+ channels. J Biol Chem, 274, 16973-16978.
Naray-Fejes-Toth, A., Fejes-Toth, G., Volk, K. A., and Stokes, J. B. (2000). SGK is a primary glucocorticoid-induced gene in the human. J Steroid Biochem. Mol Biol, 75, 51-56.
Park, J., Leong, M. L., Buse, P., Maiyar, A. C., Firestone, G. L., and Hemmings, B. A. (1999). Serum and glucocorticoid-inducible kinase (SGK) is a target of the PI 3-kinase-stimulated signaling pathway. EMBO J, 18, 3024-3033.
Perrotti, N., He, R. A., Phillips, S. A., Haft, C. R., and Taylor, S. I. (2001). Activation of serum- and glucocorticoid-induced protein kinase (Sgk) by cyclic AMP and insulin. J Biol Chem, 276, 9406-9412.
Richards, J. S., Fitzpatrick, S. L., Clemens, J. W., Morris, J. K., Alliston, T., and Sirois, J. (1995). Ovarian cell differentiation: a cascade of multiple hormones, cellular signals, and regulated genes. Recent Prog. Horm. Res, 50, 223-254.
Rozansky, D. J., Wang, J., Doan, N., Purdy, T., Faulk, T., Bhargava, A., Dawson, K., and Pearce, D. (2002). Hypotonic induction of SGK1 and Na+ transport in A6 cells. Am. J. Physiol Renal Physiol, 283, F105-F113.
Shenolikar, S, and Weinman, E. J. (2001). NHERF: targeting and trafficking membrane proteins. Am. J. Physiol Renal Physiol, 280, F389-F395.
   Ref ID: 16
Shigaev, A., Asher, C., Lafter, H., Garty, H., and Reuveny, E. (2000). Regulation of sgk by aldosterone and its effects on the epithelial Na(+) channel. Am. J. Physiol Renal Physiol, 278, F613-F619.
Shukunami, C., Shigeno, C., Atsumi, T., Ishizeki, K., Suzuki, F., and Hiraki, Y. (1996). Chondrogenic differentiation of clonal mouse embryonic cell line ATDC5 in vitro: differentiation-dependent gene expression of parathyroid hormone (PTH)/PTH-related peptide receptor. J Cell Biol, 133, 457-468.
Shukunami, C., lshizeki, K., Atsumi, T., Ohta, Y., Suzuki, F., and Hiraki, Y. (1997). Cellular hypertrophy and calcification of embryonal carcinoma-derived chondrogenic cell line ATDC5 in vitro, J Bone Miner. Res, 12, 1174-1188.
Snyder, P. M., Olson, D. R., and Thomas, B. C. (2002). Serum and glucocorticoid-regulated kinase modulates Nedd-4-2-mediated inhibition of the epithelial Na+ channel. J Biol Chem, 277, 5-8.
Staub, O., Gautschi, I., Ishikawa, T., Breitschopf, K., Ciechanover, A., Schild, L., and Rotin, D. (1997). Regulation of stability and function of the epithelial Na⁺ channel (ENaC) by ubiquitination. EMBO J, 16, 6325-6336.
Vincent, G. M. (1998). The molecular genetics of the long QT syndrome: genes causing fainting and sudden death. Annu. Rev. Med, 49, 263-274.
Wagner, C. A., Broer, A., Albers, A., Gamper, N., Lang, F., and Broer, S. (2000). The heterodimeric amino acid transporter 4F2hc/LAT1 is associated in Xenopus oocytes with a non-selective cation channel that is regulated by the serine/threonine kinase sgk-1. J Physiol, 526 Pt 1, 35-46.
Wagner, C. A., Ott, M., Klingel, K., Beck, S., Melzig, J., Friedrich, B., Wild, K. N., Broer, S., Moschen, I., Albers, A., Waldegger, S., Tummler, B., Egan, M. E., Geibel, J. P., Kandolf, R., and Lang, F. (2001). Effects of the serine/threonine kinase SGK1 on the epithelial Na(+) channel (ENaC) and CFTR: implications for cystic fibrosis. Cell Physiol Biochem., 11, 209-218.
Wald, H., Garty, H., Palmer, L. G., and Popovtzer, M. M. (1998). Differential regulation of ROMK expression in kidney cortex and medulla by aldosterone and potassium. Am. J. Physiol, 275, F239-F245.
Waldegger, S., Barth, P., Raber, G., and Lang, F. (1997). Cloning and characterization of a putative human serine/threonine protein kinase transcriptionally modified during anisotonic and isotonic alterations of cell volume. Proc. Natl. Acad. Sci. U.S.A, 94, 4440-4445.
Waidegger, S., Gabrysch, S., Barth, P., Fillon, S., and Lang, F. (2000). h-sgk serine-threonine protein kinase as transcriptional target of p 38/MAP kinase pathway in HepG2 human hepatoma cells. Cell Physiol Biochem., 10, 203-208.
Waldegger, S., Klingel, K., Barth, P., Sauter, M., Rfer, M. L., Kandolf, R., and Lang, F. (1999). h-sgk serine-threonine protein kinase gene as transcriptional target of transforming growth factor beta in human intestine. Gastroenterology, 116, 1081-1088.
Wang, J., Barbry, P., Maiyar, A. C., Rozansky, D. J., Bhargava, A., Leong, M., Firestone, G. L., and Pearce, D. (2001). SGK integrates insulin and mineralocorticoid regulation of epithelial sodium transport. Am. J. Physiol Renal Physiol, 280, F303-F313.
Wang, W. (1999). Regulation of the ROMK channel: interaction of the ROMK with associate proteins. Am. J. Physiol, 277, F826-F831.
Warntges, S., Friedrich, B., Henke, G., Duranton, C., Lang, P. A., Waldegger, S., Meyermann, R., Kuhl, D., Speckmann, E. J., Obermuller, N., Witzgall, R., Mack, A. F., Wagner, H. J., Wagner, A., Broer, S., and Lang, F. (2002a). Cerebral localization and regulation of the cell volume-sensitive serum- and glucocorticoid-dependent kinase SGK1. Pflugers Arch, 443, 617-624.
Warntges, S., Klingel, K., Weigert, C., Fillon, S., Buck, M., Schleicher, E., Rodemann, H. P., Knabbe, C., Kandolf, R., and Lang, F. (2002b). Excessive transcription of the human serum and glucocorticoid dependent kinase hSGK1 in lung fibrosis. Cell Physiol Biochem., 12, 135-142.
Webster, M. K., Goya, L., and Firestone, G. L. (1993a). Immediate-early transcriptional regulation and rapid mRNA turnover of a putative serine/threonine protein kinase. J Biol Chem, 268, 11482-11485.
Webster, M. K., Goya, L., Ge, Y., Maiyar, A. C., and Firestone, G. L. (1993b). Characterization of sgk, a novel member of the serine/threonine protein kinase gene family which is transcriptionally induced by glucocorticoids and serum. Mol Cell Biol, 13, 2031-2040.
Wulff, P., Valion, V., Huang, D. Y., Volkl, H., Yu, F., Richter, K., Jansen, M., Schlunz, M., Klingel, K., Loffing, J., Kauselmann, G., Bosl, M. R., Lang, F., and Kuhl, D. (2002). Impaired renal Na(+) retention in the sgkl-knockout mouse. J Clin Invest, 110, 1263-1268.
Yun, C. C. (2003). Concerted roles of SGK1 and the Na+/H+ exchanger regulatory factor 2 (NHERF2) in regulation of NHE3. Cell Physiol Biochem., 13, 29-40.
Yun, C. C., Chen, Y., and Lang, F. (2002). Glucocorticoid activation of Na(+)/H(+) exchanger isoform 3 revisited. The roles of SGK1 and NHERF2. J Biol Chem, 277, 7676-7683.

### Literature on Standard Methods:

Unless indicated otherwise, the laboratory methods described herein are or can be carried out in accordance with the standard literature detailed below.
Bancroft et al., 1999; Methods in Microbiology 28: 67-82.
Sambrook et al. (1989) Molecular Cloning: A Laboratory Manual. Second edition. Cold Spring Harbor Laboratory Press, Cold Spring Harbor, N.Y. 545 pp;
Current Protocols in Molecular Biology; regularly updated, e.g. Volume 2000; Wiley & Sons, Inc; Editors: Fred M. Ausubel, Roger Brent, Robert Eg. Kingston, David D. Moore, J. G. Seidman, John A. Smith, Kevin Struhl.
Current Protocols in Human Genetics; regularly uptdated; Wiley & Sons, Inc; Editors: Nicholas C. Dracopoli, Honathan L. Haines, Bruce R. Korf, Cynthia C. Morton, Christine E. Seidman, J. G. Seigman, Douglas R. Smith.
Current Protocols in Protein Science; regularly updated; Wiley & Sons, Inc; Editors; John E. Coligan, Ben M. Dunn, Hidde L. Ploegh, David W. Speicher, Paul T. Wingfield.
Molecular Biology of the Cell; third edition; Alberts, B., Bray, D., Lewis, J., Raff, M., Roberts, K., Watson, J. D.; Garland Publishing, Inc. New York & London, 1994;
Short Protocols in Molecular Biology, 5th edition, by Frederick M. Ansubel (Editor), Roger Brent (Editor), Robert E. Kingston (Editor), David D. Moore (Editor), J. G.
Seidman (Editor), John A. Smith (Editor), Kevin Struhl (Editor), October 2002, John Wiley & Sons, Inc., New York”
Gene Targeting: a practical approach (1995), Editor: A. L. Joyner, IRL Press.
Transgenic Animal Technology A Laboratory Handbook. C. A. Pinkert, editor; Academic Press Inc., San Diego, Calif., 1994 (ISBN: 0125571658).

The invention is explained in detail below by means of examples and figures which are not, however, intended to restrict the subject matters of the present invention:

### EXAMPLE 1

### Gene Expression Analyses Using Osteoarthrosis Arrays

The gene expression analyses were based on an osteoarthrosis-specific cDNA array which was produced to order by GPC-Biotech and consists of 4500 cartilage-relevant genes which have been applied to nylon membranes. As probe for hybridization of the array, total RNA (preparation by standard methods) from healthy and osteoarthrotic articular cartilage was translated by reverse transcription with incorporation of ³²P-labeled nucleotides into a radioactive cDNA and incubated with the cDNA samples of the array by standard methods. The hybridization and subsequent washing of the membranes was followed by detection of the hybridization signals by autoradiography. 270 of the genes which were expressed differentially differently between healthy and osteoarthrotic cartilage were considered for further analyses. One of these genes was surprisingly SKG: The findings of the inventors' gene expression analysis showed for the first time that SGK expression is induced in osteoarthrosis in articular cartilage. It was possible to confirm this finding by the number of clones in cDNA libraries: it emerged from this that, in contrast to libraries of normal cartilage, SGK cDNA is expressed 14 times as frequently in cDNA libraries of osteoarthrotic cartilage.

### EXAMPLE 2

### Preparation of the Arrays and Hybridization

The clones for preparing the arrays were selected by SSH (subtractive suppression hybridization). Also used were control clones and clones of housekeeping genes. The amplification took place with use of a PCR kit from Qiagen, Hilden UK in 384-well polypropylene plates (Genetix, UK) using the M13 forward (5′ GCT ATT ACG CCA GCT GGC GAA AGG GGG ATG TG 3′) and M13 reverse primer (5′ CCC CAG GCT TTA CAC TTT ATG CTT CCG GCT CG 3′) in a PCR reaction with a reaction volume of 50 μl as stated by the manufacturer (Qiagen, Hilden UK, see above). The reaction mixtures were inoculated, using a 384-pin transfer apparatus (Genetix), with 2 μl or less of bacterial culture, and the plates were then sealed with plastic film (Genetix). The PCR amplification was carried out in an automated waterbath system (KBioSystems, Basildon, UK) under standard conditions (Meier-Ewert, S. et al.). The PCR products from 4396 cDNA clones were transferred singly to 8×12 cm replica nylon membranes (Hybond N+, Amersham Pharmacia Biotech) using a robotic applicator as described by Maier et al., and then immobilized on the membrane by air-drying. The PCR products were moreover applied to the membranes twice in the form of a 5×5 pattern with a point-to-point distance of 850 μm.

### EXAMPLE 3

### Probe Labeling, Hybridization and Washing

A total amount of 2 μg of total RNA were transcribed into cDNA using superscript II reverse transcriptase (#18064-071, Invitrogen) in four different reaction mixtures. The mixtures each comprised in this case: 200 units of superscript II (#18064-071, Invitrogen), 50 μCi of ³³P α-dCTP (#NEG313H, NEN Life Science Products GmbH) in each case 0.66 mM dATP, dGTP, dTTP, 0.5 μg of random hexamers (Amersham Pharmacia Biotech) and 10 mM DTT in superscript II first-strand reaction buffer (Invitrogen). The reaction was incubated at 42° C. for 4 hours and then the cDNA was purified using quick spin columns (Roche, Penzberg Germany) as stated by the manufacturer. The hybridization was preceded by denaturation of each probe using 0.11 volumes of denaturing solution (1 M NaOH/1 mM EDTA) and 5 μg of human COT-1 DNA (15279-011, Invitrogen) at 70° C. for 20 minutes. Each probe was then neutralized by adding one volume quantity of neutralizing solution (1 M Na phosphates/pH 7.0) at 70° C. for 10 minutes and then added to the hybridization mixture. Reverse-transcribed cDNA from cartilage total RNA was employed as probe.

For each hybridization experiment, 3 arrays were prehybridized in 50 ml of hybridization buffer (7% SDS, 50% deionized formamide, 5×SSC, 50 mM Na phosphate buffer pH 7.0 and 2% dry milk powder) for 2 hours before the probe was added. The hybridization took place after addition of the probe in plastic vessels (20 cm×10 cm) at 50° C. for 16 hours. After the hybridization, the arrays were washed twice in a 2×SSC, 1% SDS buffer and twice in a 0.1% SSC and 0.5% SDS buffer. Each washing step was carried out at 65° C. for 15 minutes. The membranes were then packed in clingfilm, put into contact with phospho-storage screens for 5 days and scanned using a FUJI BAS 5000 imaging system with a resolution of 25 μm. The images were analyzed using a commercially available array analysis program (VisualGrid, GPC Biotech AG) which converted the average signal intensity of each position applied to the array into computer-readable data and stored it as file in a storage medium.

### EXAMPLE 4

### Data Analysis

Raw data in the files which the signal intensity of the various array signals (which each corresponded to individual clones) were analyzed as follows: initially, the background signal was corrected by subtracting signal intensities derived from positions not loaded with DNA. Subsequently, average signal intensities were corrected taking account of the six different replica values per clone (3 arrays with in each case two data points) and logarithmically transformed, and standard deviations, were determined for each of the clones on the array. Then possible samples outliers? were identified by combining data from individual patient's samples using a hierarchical clustering algorithm based on the average-linkage method (Eisen et al., 1998). Finally, differentially expressed clones were identified by comparing the resulting average signal intensities (comparison of the average signal intensity of a particular clone in a cDNA sample from cartilage of OA patients with the average signal intensity in a cDNA sample from healthy cartilage).

Alternatively, the average signal intensity of a particular clone of each group of OA patients was compared with the average signal intensity of the samples of healthy cartilage.

The level of expression of a gene in a particular tissue sample represented by a particular cDNA clone in each case was related to the average signal intensity of a particular clone from the hybridization experiment by employing probes which corresponded to the tissue samples. A higher average signal intensity in this case corresponded to a higher level of expression. The significance of gene expression was determined for each comparison by using a two-tailed T test. A p value of 0.1 was employed as significance threshold in order thus to identify clones which represent genes which were differentially expressed in a group of samples from OA patients compared with normal cartilage samples.

### EXAMPLE 5

### Hybridization of the cDNA Arrays

To confirm the differential gene expression, individual clones identified by the abovementioned procedure were employed as probes and hybridized with arrays onto which primary cDNA libraries from osteoarthrotic and normal tissue were immobilized. The primary cDNA libraries were in this case generated by standard methods, introduced into bacteria, a selection of bacterial clones which represents the library was isolated, and this selection of clones was introduced into microtiter plates by standard methods (see Bancroft et al., 1999). (1999; Methods in Microbiology 28: 67-82). The arrays were produced by the methods described above using the robotic systems described above but were in each case 23×23 cm in size and comprised about ˜40 000 PCR products which corresponded to primary cDNA clones applied in duplicates. Two sets of arrays were employed for these experiments; a first set of 5 arrays corresponding to 200 000 cDNA clones of a cDNA library from normal tissue and a second set of 5 arrays corresponding to 200 000 cDNA clones of a cDNA library from osteoarthotic tissue.

The PCR products of individual clones which represent differentially expressed genes were radiolabeled by random priming (DecaPrime system, Ambion) by standard methods. Each labeled clone was hybridized onto one replicate of the first and second of the two array sets, resulting a total number of 10 arrays, each of which represented 200 000 different primary cDNA clones from normal and osteoarthrotic cartilage tissue. The hybridizations were in this case carried out in plastic dishes with an area of 24×24 cm using church buffer (7% SDS, 0.5 m Na phosphate buffer, pH 7.0, 1 mM EDTA) at 65° C. for 16. The washing steps corresponded to those previously described. After the phosphor storage screens had been exposed to the hybridized arrays packed in film overnight, the arrays were scanned in accordance with the above procedure using the FUJI BAS 1800 II system at a resolution of 100 μm, and the signals were transformed into storable data. The number of primary cDNA clones on each set of arrays which hybridized with the labeled clones was determined using the aforementioned image analysis system and the procedure described previously. A particular gene with higher expression in a first tissue than in a second in this case resulted in a larger number of hybridization signals on the array set which corresponded to the primary cDNA library of the first tissue than on the array set which comprised the library of the second tissue.

### EXAMPLE 6

Investigation of the molecular mechanisms of OA by comparative gene expression analyses represents an attractive approach to the analysis of the pathological state and the identification of novel molecular points of attack for pharmacological intervention. Interpretation of the results used for this purpose from “genomics analyses” is complicated because multiple processes, in some cases in opposite directions, proceed in parallel to one another in OA-affected cartilage. An example which may be mentioned here is the simultaneous occurrence of cartilage-constructing and cartilage-degrading activities.

In order to obtain a first approximation to the potential function of a gene differentially expressed in OA compared with normal cartilage, there is a need for systems in which cartilage-degrading and -constructing activities can be visualized separately.

One example of a system suitable for this purpose is a skeleton development model, endochondral ossification. Cartilage synthesis and cartilage degradation likewise take place simultaneously during endochondral ossification. In contrast to OA, however, this takes place in zones which are clearly demarcated from one another and which also have differently differentiated cell types. The zone of proliferating cartilage cells is demarcated from the region of so-called hypertrophic cartilage cells which are clearly identifiable morphologically by their increased cell volume. Rearrangements and degradation events of the normal extracellular cartilage matrix (ECM) whose main constituents are typically composed of collagens of type II, IX and XI and the proteoglycan aggrecan take place in this region. Hypertrophic cartilage cells by contrast synthesize the type X collagen which is specific for this zone. In the hypertrophic cartilage zone there is calcification of the tissue, and loss of the antiangiogenic status of the cartilage and vascularization of the tissue occur, with subsequent formation of bone adjoining the hypertrophic cartilage zone of the skeletal primordium (FIG. 9A). Residues of cartilage ECM are still present at the site of bone formation in the early stage of development and are then further degraded or serve as differentiation matrix for other cells.

Accordingly, to visualize the activity of genes expressed under osteoarthrotic conditions in articular cartilage in such a skeletal development model, the basis must be a cartilage-constructing function for expression of the gene to be investigated in the proliferating zone and a cartilage-degrading function for expression in the hypertrophic zone or embryonic bone.

Since no data at all on expression of SGK-1 in cartilage tissue were previously known, the expression of SGK-1 mRNA, as described above, was tested during endochondral ossification. The aim thereof was to obtain initial information about the significance of the elevated SGK-1 mRNA level in cartilage tissue which was detected in the foregoing array analyses. The extremities of neonatal mice were used as tissue to be investigated. During embryonic development of mice, skeletal growth and the process of endochondral ossification is still proceeding at this time.

The SGK-1 mRNA was then investigated by means of the method of in situ hybridization.

For this purpose, the extremities of neonatal mice and mouse embryos of various stages of development were fixed overnight in 4% paraformaldehyde (PFA) which was dissolved in phosphate-buffered saline (PBS) and dehydrated by stepwise transfer into an ascending series of different ethanol concentrations (replacement of the PBS solution by ethanol/PBS solutions). The dehydrated tissues were then transferred into xylene and paraffin-embedded. For this purpose, initial incubation in a xylene/paraffin mixture was followed by the tissues then being put into pure paraffin and, after evaporation of the xylene, embedded in the wax. The tissue sections for in situ hybridization were prepared by dividing the paraffin-embedded embryos and extremities into sections 7 μM in diameter and transferring them to microscope slides. The sections were then labeled with digoxygenin-11-UTP and hybridized SGK-1-specific antisense riboprobes. The riboprobes were transcribed by standard methods using T7 RNA polymerase (Roche) from an SGK-1 cDNA fragment which was cloned into the plasmid vector pCR^{R}-bluntII-TOP^{R} (Invitrogen). The in vitro transcription was preceded by linearization of the plasmid at the end of the SGK-1 cDNA sequence with KpnI (Invitrogen) in order to terminate transcription there.

The sequence of the cDNA fragment of murine SGK-1 (nucleotides 746 to 1425) used represents a partial sequence of the sequence disclosed in the NCBI nucleotide database under BC005720 (nucleotide 746-1425) and is depicted as SEQ ID No. 13 in FIG. 8.

The in situ hybridization of the tissue sections was carried out as described by Dietz et al. (1999). After detection of the hybridization products, the sections were covered with coverslips in Kaiser's glycerin gelatine (Merck) and analyzed and photographed using a Zeiss Axioplan 2 microscope. The inventors were able, on the basis of the in situ hybridizations described above, for the first time to detect SGK-1 mRNA in cartilage cells. The expression domain of the SGK-1 mRNA detected in this case was located specifically in the hypertrophic cartilage cells (FIG. 9c) and was undetectable in the proliferating cartilage-constructing cells. It was possible to show this finding unambiguously by comparing with the occurrence of the mRNA for collagen X, the marker gene for hypertrophic cartilage cells (FIG. 9B). The proliferating cartilage was by contrast unambiguously negative in relation to SGK-1 mRNA (FIG. 9c).

It emerges from the results of the in situ hybridization experiments carried out that the natural occurrence of SGK-1 in cartilage is not connected to the synthesis and maintenance of cartilage but displays its function in transformation (hypertrophy) and degradation thereof.

Expression of SGK-1 in osteoarthrotic cartilage is accordingly a process which favors or is a partial cause of the pathology of OA. SGK-1 might, through its regulatory properties, be a new key molecule for the induction of early pathological cartilage changes as well as of later degradative activities in cartilage. For this reason, SGK-1 is a very interesting novel target molecule for pharmacological intervention in osteoarthrosis.

### EXAMPLE 7

In order to check the presumptions derived from the preceding experiments about the function of SGK-1 in cartilage, further in vitro experiments were carried out on the chondrogenic cell line ATDC5.

ATDC5 is a clonal cell line obtained from the mouse teratocarcinoma line AT805 and undergoes a cartilaginous differentiation under the influence of insulin (10 μg/ml of culture medium). For the experimental procedure, the cells are maintained as monolayer on cell culture dishes. After a confluent cell lawn has formed, condensations of cells start to form under the influence of insulin and, after a relatively long period of time, form light-refracting clusters and increase in number. This cartilaginous differentiation can easily be visualized by staining the cartilage proteoglycans with Alcian blue (Shukunami et al., 1996). In a similar manner to endochondral ossification, the cells possess a further differentiation potential to hypertrophy and calcification. The process is assisted by specific medium and carbon dioxide conditions (Shukunami et al., 1997). Calcification of the cultures can be detected by Ca²⁺ staining with alizarin red. At the molecular level it is clearly evident that there is an increase in the mRNA for collagen X, the marker gene for hypertrophic cartilage cells (Shukunami et al., 1997). In the experiments carried out here it was not possible for the hypertrophic differentiation to be separated entirely unambiguously from the chrondrocyte-proliferating activity of the ADTC5 cells so that, in parallel with the normal cartilage marker genes such as type II collagen, also small amounts of type X collagen were detectable at the mRNA level. However, these then increased markedly on induction of calcification of the cultures (FIG. 10), whereas the amounts of collagen type II mRNA diminished (no figure).

Expression of SGK-1 mRNA under these conditions was similar to collagen×(Col10a1) mRNA. During the cartilaginous differentiation and proliferation of the ATDC5 cells, basal amounts of SGK-1 mRNA were detectable and then rose drastically after hypertrophy and calcification of the cells were induced (FIG. 10). The amounts of SGK-1 and Col10a1 mRNA were detected by carrying out quantitative PCR experiments and Northern blot hybridizations. Both analysis methods provided comparable results. The findings from the in situ hybridizations were therefore confirmed in a comparable in vitro model.

### EXAMPLE 8

For targeted investigation of the function of SGK-1 during cartilage differentiation, human SGK-1 was overexpressed in ATDC5 cells by a retroviral vector system (PLPCX vector) derived from MMLV (Murine Moloney Leukemia Virus). Two different SGK-1 variants were used in this case: a kinase-deficient mutant (mutation threonine²⁵⁶ and serine⁴²² to alanine=SGK-1 KD) and the naturally occurring functional form (SGK-1 wt). ATDC5 cells which contained only the retroviral vector without SGK-1, or were untransduced, were used as control for viral SGK-1 transduction. The cells were then stimulated with insulin to cartilage differentiation and analyzed after a period of 21 days. The extent of cartilaginously differentiated cells was investigated by analyzing the proteoglycan synthesis. For this purpose, the cultures were stained with Alcian blue and tested for expression of the mRNA for aggrecan, the main proteoglycan of cartilage (FIG. 11).

It was possible to show clearly in these experiments that overexpression of SGK-1 brings about inhibition of cartilage synthesis. Both the amount of Alcian blue-stained proteoglycan (FIG. 11) and of aggrecan mRNA (FIG. 12) was significantly reduced. The kinase-deficient SGK-1 form by contrast had no negative effect on this parameter (FIG. 11, 12). The described controls showed similarly neutral behavior. It was thus possible to show that inhibition of cartilage synthesis and ECM formation is attributable to the effect of SGK-1 and was not produced by the artificially increased amount of protein as a result of overexpression.

Various conclusions can be drawn from these experiments about the effect of SGK-1 in articular cartilage affected by OA.

Firstly, SGK-1-expressing cartilage cells are no longer able to synthesize sufficient extracellular matrix such as, for example, proteoglycans which are essential for the function of the tissue. Secondly, the cartilage cells are prevented from compensating for or repairing degradation processes by increasing the expression of genes such as aggrecan. The function of SGK-1 as potential initiator and central factor of OA pathology is thus confirmed.

SGK-1 therefore represents a highly interesting target molecule for finding and developing novel active ingredients for the treatment of degenerative cartilage changes, especially osteoarthrosis.

Expression of SGK-1, -2 and -3 in articular cartilage cells ought to provide important information for further analysis of the function of the molecules. It is possible for this purpose for example to produce and analyze transgenic and knockout models of the mouse by standard methods known to the skilled worker (see, for example, the listed literature on standard methods).

### DESCRIPTION OF THE FIGURES

FIG. 1

Coding polynucleotide sequence of human serum glucocorticoid-regulated kinase 1 (SGK1) according to NCBI access number NM_{—}005627, AF153609 (SEQ ID No. 1).

FIG. 2

Coding polynucleotide sequence of human serum glucocorticoid-regulated kinase 2 (SGK2) according to NCBI access number AF169034, AF186470 (SEQ ID No. 2).

FIG. 3

Coding polynucleotide sequence of human serum glucocorticoid-regulated kinase 3 (SGK3) according to NCBI access number NM_{—}013257, AF085233, AF169035 (SEQ ID No. 3).

FIG. 4

Amino acid sequence of human serum glucocorticoid-regulated kinase 1 (SGK1) according to NCBI access number NP_{—}005618 (SEQ ID No. 4).

FIG. 5

Amino acid sequence of human serum glucocorticoid-regulated kinase 2 (SGK2) according to NCBI access number NP-733794, NP_{—}057360 (SEQ ID No. 5). In the figure, the first amino acids of the two different SGK 2 isoforms α and β are emboldened. The DDG/EMBL/GenBank access numbers of these two subtypes are AF169034 (α) and AF186470 (β). SGK2α is a protein of 367 amino acids, while SGK2β is a protein of 427 amino acids.

FIG. 6

Amino acid sequence of human serum glucocorticoid-regulated kinase 3 (SGK3) according to NCBI access number Q96BR1 (SEQ ID No. 6). SGK3 is a protein with a length of 429 amino acids.

FIG. 7

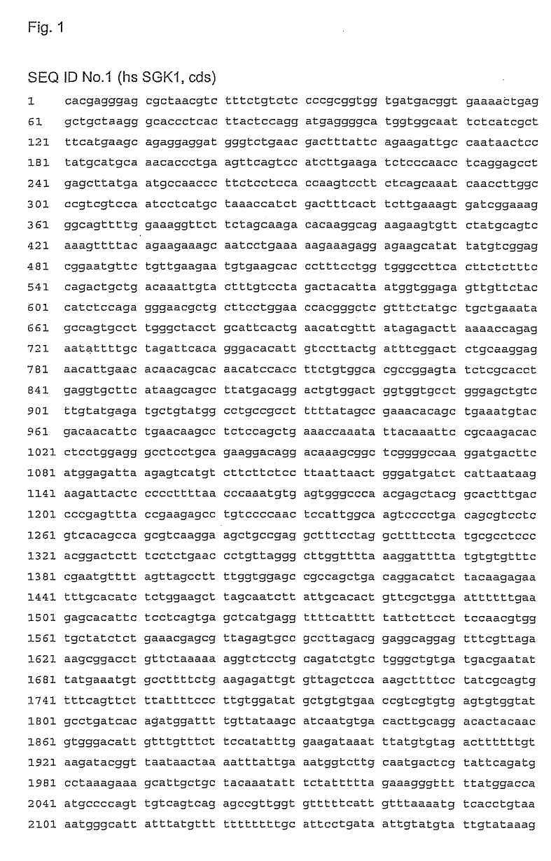

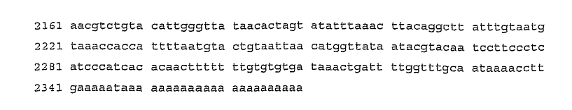

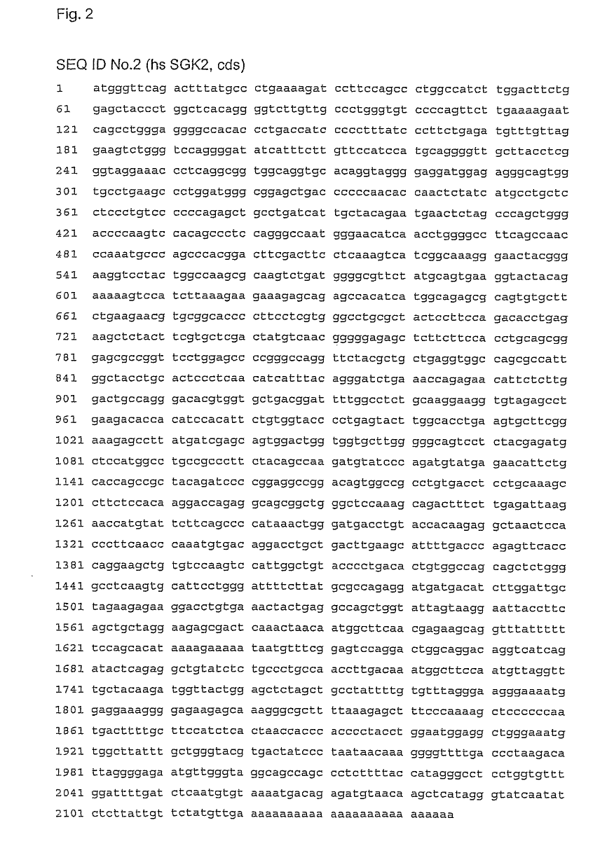

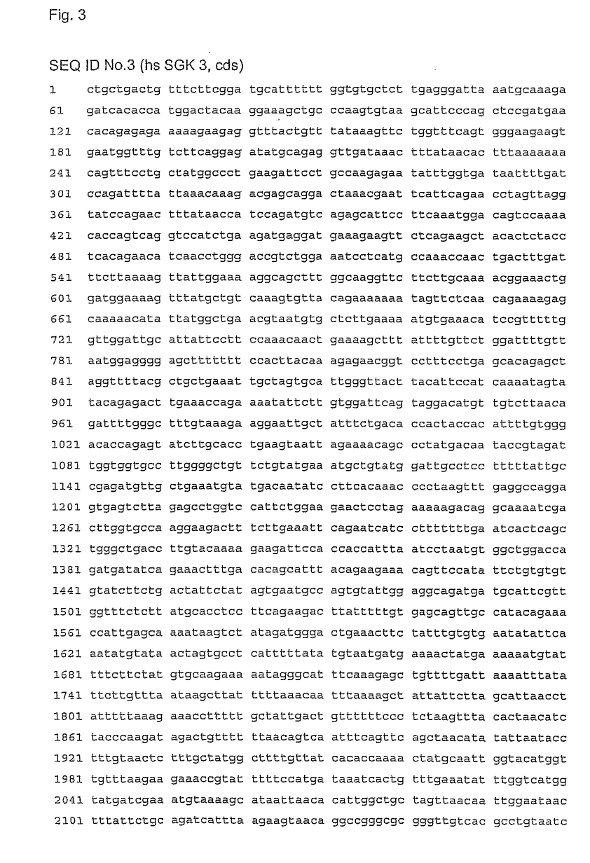

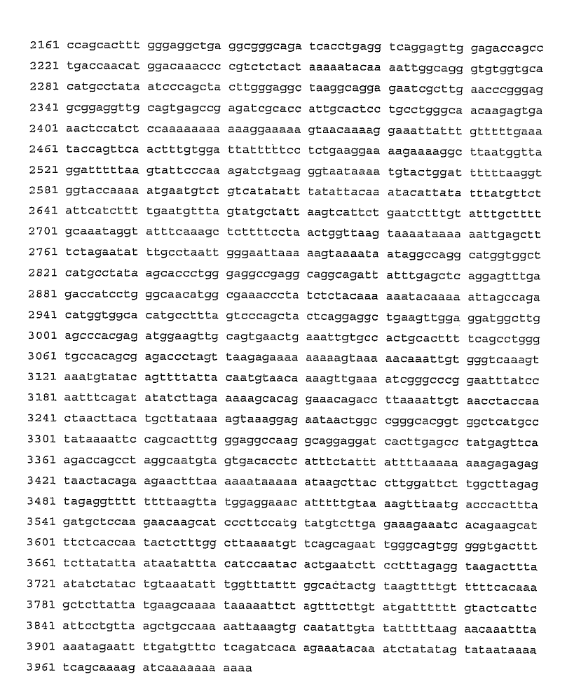

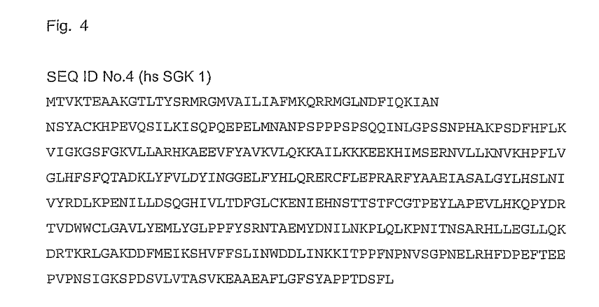

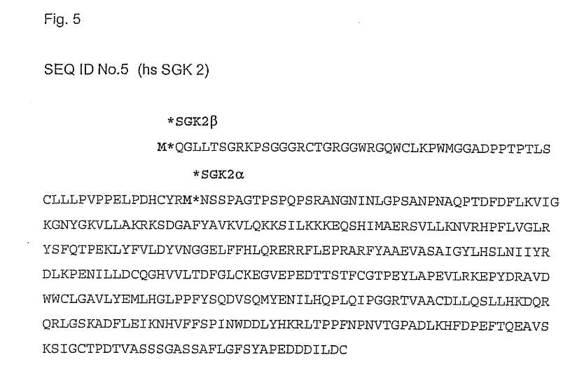

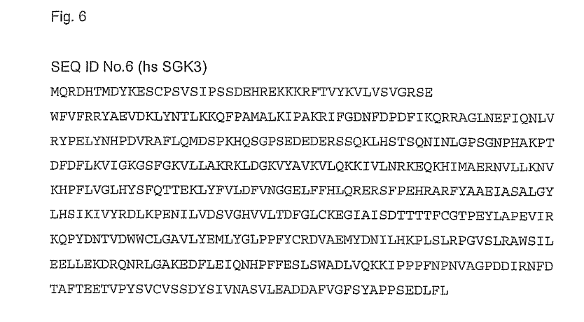

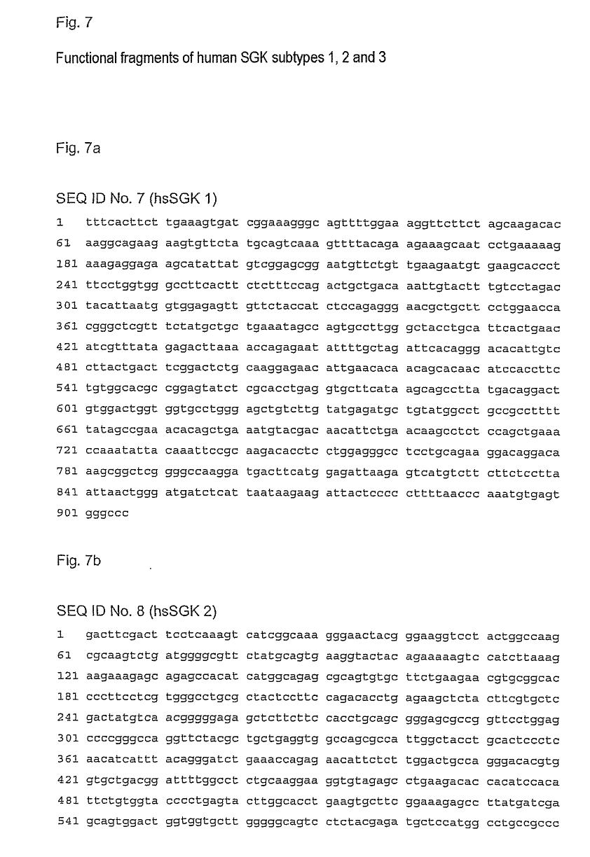

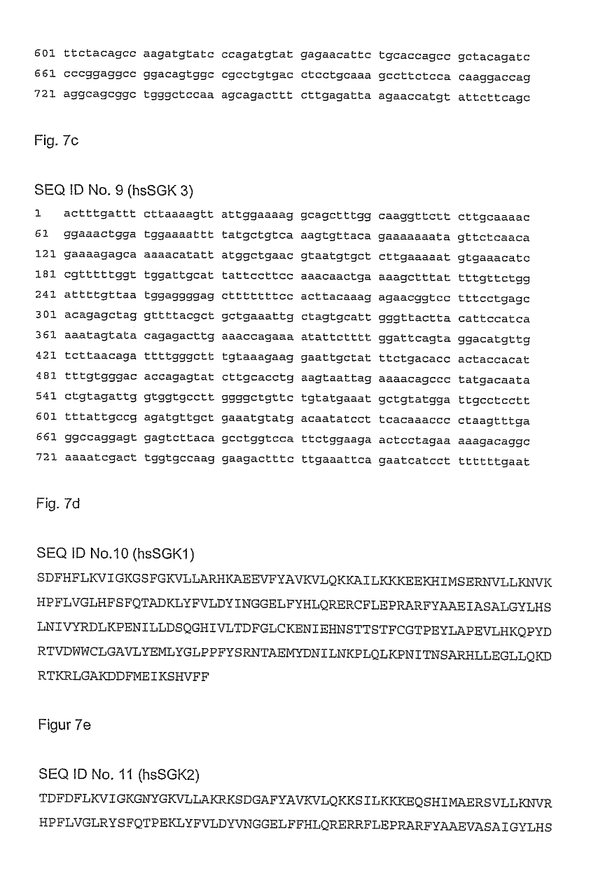

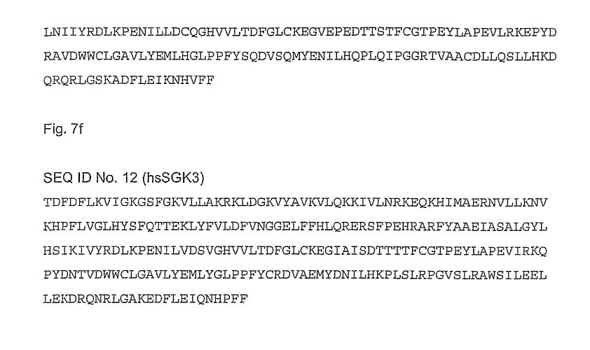

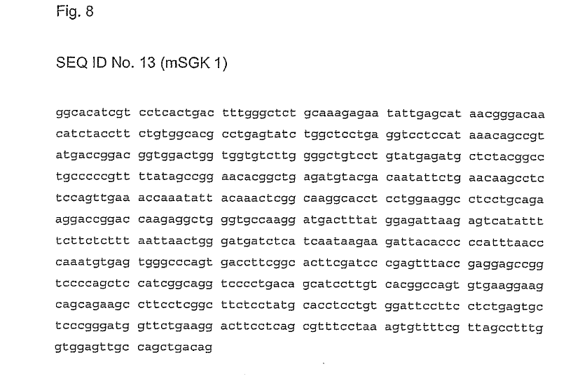

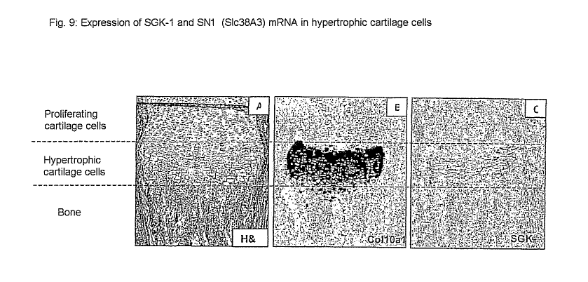

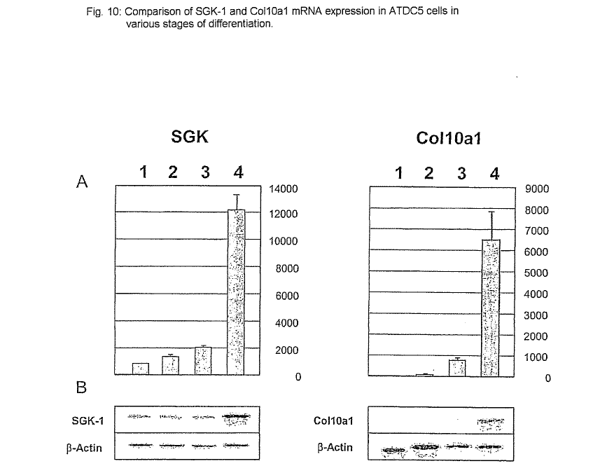

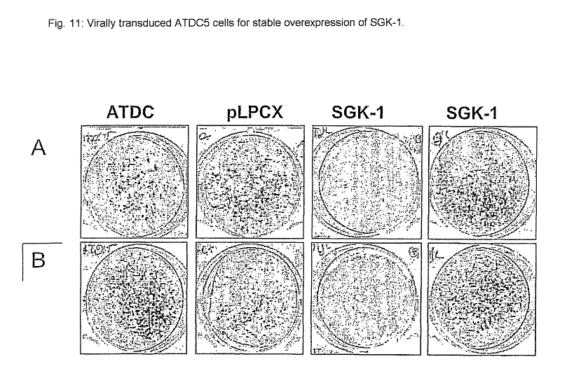

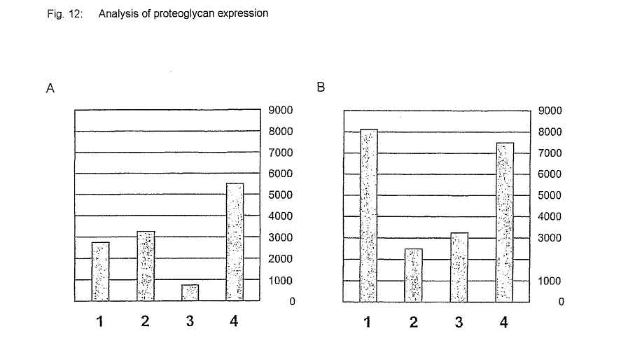

Amino acid and encoding polynucleotide sequences of functional fragments of SGK subtypes 1 to 3: FIG. 7A shows a fragment containing the kinase domain of the SGK1-encoding polynucleotide sequence (SEQ ID No. 7); FIG. 7B shows a fragment containing the kinase domain of the SGK2-encoding polynucleotide sequence (SEQ ID NO. 8); FIG. 7C shows a fragment containing the kinase domain of the SGK3-encoding polynucleotide sequence (SEQ ID No. 9); FIG. 7D shows a fragment containing the kinase domain of the SGK1 amino acid sequence (SEQ ID No. 10); FIG. 7E shows a fragment containing the kinase domain of the SGK3 amino acid sequence (SEQ ID No. 11); FIG. 7F shows a fragment containing the kinase domain of the SGK3 amino acid sequence (SEQ ID NO. 12);

FIG. 8

cDNA fragment of the mouse SGK-1 mRNA:

FIG. 8 shows the nucleotide sequence of a cDNA fragment which was cloned from the mouse SGK-1 mRNA and used to prepare riboprobes (SEQ ID No. 13).

FIG. 9

Detection of SGK-1 mRNA expression by in situ hybridization on tissue sections of the tibia of a neonatal mouse.
A: Control staining with hematoxilin and eosin.
B: Detection of Col10a1 expression in hypertrophic cartilage cells by in situ hybridization with digoxigenin-labeled antisense riboprobes and subsequent color reaction. C: Detection of SGK-1 mRNA mRNA in hypertrophic cartilage cells by in situ hybridization with digoxigenin-labeled antisense riboprobes and subsequent color reaction.

The illustrations in the figure showed the detection of SGK-1 (FIG. 9C) in the hypertrophic cartilage cells of the tibia of a neonatal mouse by in situ hybridization of specific antisense riboprobes. For better identification of the cells, a corresponding control staining of the tissue section is shown (FIG. 9A).

FIG. 10

Comparison of SGK-1 and Col10a1 mRNA expression in ATDC5 cells in various stages of differentiation.
1: ATDC5 cells in the subconfluent stage
2: Confluent ATDC5 cells
3: Chondrogenically differentiated ATDC5 cells after 3-week insulin addition
4: Chondrogenically differentiated ATDC5 cells as described for 3. after additional calcification over a period of two weeks.

FIG. 10A shows the analysis of amounts of SGK-1 and Col10a1 mRNA by quantitative PCR. Numerical units are stated to indicate the extent of the expression amplitudes.

FIG. 10B shows the detection of SGK-1 and Col10a1 mRNAs by Northern blot hybridization.

The expression of β-actin mRNA was measured in A and B to calibrate for the amounts of total cellular RNA employed in the reactions.

FIG. 11

Virally transduced ATDC5 cells for stable overexpression of SGK-1.

| | |
|---|---|
| ATDC5: | untreated cells as control |
| pLCPCX: | stable integration of the transduction vector without |
| | SGK-1, likewise for control |
| SGK-1 wt: | overexpression of functional SGK-1 |
| SGK-1 KD: | overexpression of a kinase-deficient SGK-1 |

The cells were maintained under chondrogenic differentiation conditions for 21 days after reaching confluence. The extent of chondrogenic differentiation and proteoglycan production was detected by staining with Alcian blue.

SGK-1-overexpressing cells show significantly reduced amounts of cartilaginously differentiated cells and proteoglycan formation, whereas the kinase-deficient SGK-1 has no effect in this regard.

A and B shows the identical test in two independent experiments.

FIG. 12

Analysis of proteoglycan expression of the samples described in FIG. 11 at the level of aggrecan mRNA by quantitative PCR.

A and B correspond to the samples depicted in FIGS. 11A and B.
1. ATDC5
2. pLPCX
3. SGK-1 wt
4. SGK-1 KD

Numerical units are stated to indicate the extent of the expression amplitudes. The amounts of measured aggrecan mRNA almost all show parallel behavior to the amounts shown in FIG. 11 for the proteoglycan detected by Alcian blue staining.

Only the pLPCX sample in FIG. 11B shows in the comparison at the mRNA level a smaller amplitude compared with the SGK-1 wt sample. However, the greatly reduced aggrecan mRNA expression after overexpression of SGK-1 wt compared with ATDC5 and SGK-1 KD is unambiguous.

1-22. (canceled)

23. A method for identifying an active ingredient for the prevention or treatment of degenerative cartilage disorders comprising the steps consisting of
i. contacting a polypeptide consisting of an SGK protein, functional derivative or fragment thereof with a test active ingredient,
ii. measuring SGK activity in the presence of said test active ingredient,
iii. measuring SGK activity in the absence of said active ingredient,
iv. and comparing the SGK activity in step ii and iii and determining whether said SGK activity is lower in the presence of said test active ingredient than in its absence.

24. A method for identifying an active ingredient for the prevention or treatment of degenerative cartilage disorders comprising the steps consisting of
i. contacting a nucleic acid encoding a SGK protein, derivative or fragment thereof with a test active ingredient in a transcription assay,
ii. measuring the amount of expression of a mRNA encoding a SGK protein, derivative or fragment thereof in the presence of said test active ingredient,
iii. measuring the amount of expression of a mRNA encoding a SGK protein, derivative or fragment thereof in the absence of said test active ingredient,
iv. and comparing the amount of mRNA expression with and without test active ingredient to determine whether said mRNA is lower in the presence of said test active ingredient than in its absence.

25. At method for identifying active ingredients for the prevention or treatment of degenerative cartilage disorders comprising the steps consisting of
i. contacting a nucleic acid sequence encoding a SGK protein, a functional derivative or fragment thereof with a test active ingredient in translation system,
ii. measuring the expression of SGK protein, functional derivative or fragment thereof in the presence of said test active ingredient;
iii. measuring the expression (if SGK protein, functional derivative or fragment thereof in the absence of said test active ingredient and
iv. and comparing the protein expression with and without said test active ingredient to determine whether protein expression is lower in the presence of said test active ingredient than in its absence.

26. The method according to claims 23, 24 and 25 in the alternative that is calibrated as a high-throughput screening assay.

27. A method for identifying a degenerative cartilage in a cartilage sample comprising the steps of
i. measuring the content of SGK protein or SGK mRNA in an isolated cartilage sample,
ii. and comparing said content of SGK protein or SGK mRNA with an isolated sample of a known non-degenerated cartilage.

28. The method according to claim 23, 24 or 25 in the alternative, wherein said SGK is SGK1, 2 or 3.

29. The method of claim 24 or 25 wherein the polynucleotide comprises the group consisting of
i. SEQ ID No. 1, SEQ ID No. 2 or SEQ ID No. 3,
ii. a sequence which hybridizes with SEQ ID No. 1, SEQ ID No. 2 or SEQ ID No. 3, under stringent conditions and codes for a polypeptide having SGK function,
iii. a sequence which, on the basis of the genetic code, is degenerate in relation to one of the sequences according to i. or ii. and codes for a polypeptide having SGK function and
iv. a fragment of i. or ii, or iii. which codes for a polypeptide having SGK function.

30. The method of claim 24 or 25 wherein the polynucleotide comprises the group consisting of
i. SEQ ID No. 7, SEQ ID No. 8 or SEQ ID No. 9,
ii. a sequence witch hybridizes with SEQ ID No. 7, SEQ ID No. 8 or SEQ ID No. 9, under stringent conditions and codes for a polypeptide having SGK function,
iii. a sequence which, on the basis of the genetic code, is degenerate in relation to one of the sequences according to i. or ii. and codes for a polypeptide having SGK function and
iv. a fragment of i. or ii. or iii. which codes for a polypeptide having SGK function.

31. the method according to claim 23 wherein the polypeptide comprises the group consisting of SEQ ID No. 4, SEQ ID No. 5, SEQ ID No. 6, SEQ ID No. 10, SEQ ID No. 11 or SEQ ID No. 12.


SEQUENCE LISTING (13 sequences)

SEQ ID NO: 1 - DNA, 2370 bp, Homo sapiens
cacgagggagcgctaacgtctttctgtctccccgcggtggtgatgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgatttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgagctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaattttttgaagagcacattctcctcagtgagctcatgaggttttcatttttattcttccttccaacgtggtgctatctctgaaacgagcgttagagtgccgccttagacggaggcaggagtttcgttagaaagcggacctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcctaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaataaaaaaaaaaaaaaaaaaaaaaa

SEQ ID NO: 2 - DNA, 2146 bp, Homo sapiens
atgggttcagactttatgccctgaaaagatccttccagccctggccatcttggacttctggagctaccctggctcacaggggtcttgttgccctgggtgtccccagttcttgaaaagaatcagcctgggaggggccacaccctgaccatccccctttatcccttctgagatgtttgttaggaagtctgggtccaggggatatcatttcttgttccatccatgcaggggttgcttacctcgggtaggaaaccctcaggcggtggcaggtgcacaggtaggggaggatggagagggcagtggtgcctgaagccctggatgggcggagctgaccccccaacaccaactctatcatgcctgctcctccctgtccccccagagctgcctgatcattgctacagaatgaactctagcccagctgggaccccaagtccacagccctccagggccaatgggaacatcaacctggggccttcagccaacccaaatgcccagcccacggacttcgacttcctcaaagtcatcggcaaagggaactacgggaaggtcctactggccaagcgcaagtctgatggggcgttctatgcagtgaaggtactacagaaaaagtccatcttaaagaagaaagagcagagccacatcatggcagagcgcagtgtgcttctgaagaacgtgcggcaccccttcctcgtgggcctgcgctactccttccagacacctgagaagctctacttcgtgctcgactatgtcaacgggggagagctcttcttccacctgcagcgggagcgccggttcctggagccccgggccaggttctacgctgctgaggtggccagcgccattggctacctgcactccctcaacatcatttacagggatctgaaaccagagaacattctcttggactgccagggacacgtggtgctgacggattttggcctctgcaaggaaggtgtagagcctgaagacaccacatccacattctgtggtacccctgagtacttggcacctgaagtgcttcggaaagagccttatgatcgagcagtggactggtggtgcttgggggcagtcctctacgagatgctccatggcctgccgcccttctacagccaagatgtatcccagatgtatgagaacattctgcaccagccgctacagatccccggaggccggacagtggccgcctgtgacctcctgcaaagccttctccacaaggaccagaggcagcggctgggctccaaagcagactttcttgagattaagaaccatgtattcttcagccccataaactgggatgacctgtaccacaagaggctaactccacccttcaacccaaatgtgacaggacctgctgacttgaagcattttgacccagagttcacccaggaagctgtgtccaagtccattggctgtacccctgacactgtggccagcagctctggggcctcaagtgcattcctgggattttcttatgcgccagaggatgatgacatcttggattgctagaagagaaggacctgtgaaactactgaggccagctggtattagtaaggaattaccttcagctgctaggaagagcgactcaaactaacaatggcttcaacgagaagcaggtttattttttccagcacataaaagaaaaataatgtttcggagtccaggactggcaggacaggtcatcagatactcagaggctgtatctctgccctgccaaccttgacaaatggcttccaatgttaggtttgctacaagatggttactggagctctagctgcctattttgtgtttagggaagggaaaatggaggaaaggggagaagagcaaagggcgcttttaaagagctttcccaaaagctccccccaatgacttttgcttccatctcactaaccacccacccctacctggaatggaggctgggaaatgtggcttatttgctgggtacgtgactatccctaataacaaaggggttttgaccctaagacattaggggagaatgttgggtaggcagccagccctcttttaccatagggcctcctggtgtttggattttgatctcaatgtgtaaaatgacagagatgtaacaagctcatagggtatcaatatctcttattgttctatgttgaaaaaaaaaaaaaaaaaaaaaaaaaaa

SEQ ID NO: 3 - DNA, 3984 bp, Homo sapiens
ctgctgactgtttcttcggatgcattttttggtgtgctcttgagggattaaatgcaaagagatcacaccatggactacaaggaaagctgcccaagtgtaagcattcccagctccgatgaacacagagagaaaaagaagaggtttactgtttataaagttctggtttcagtgggaagaagtgaatggtttgtcttcaggagatatgcagaggttgataaactttataacactttaaaaaaacagtttcctgctatggccctgaagattcctgccaagagaatatttggtgataattttgatccagattttattaaacaaagacgagcaggactaaacgaattcattcagaacctagttaggtatccagaactttataaccatccagatgtcagagcattccttcaaatggacagtccaaaacaccagtcaggtccatctgaagatgaggatgaaagaagttctcagaagctacactctacctcacagaacatcaacctgggaccgtctggaaatcctcatgccaaaccaactgactttgatttcttaaaagttattggaaaaggcagctttggcaaggttcttcttgcaaaacggaaactggatggaaaagtttatgctgtcaaagtgttacagaaaaaaatagttctcaacagaaaagagcaaaaacatattatggctgaacgtaatgtgctcttgaaaaatgtgaaacatccgtttttggttggattgcattattccttccaaacaactgaaaagctttattttgttctggattttgttaatggaggggagctttttttccacttacaaagagaacggtcctttcctgagcacagagctaggttttacgctgctgaaattgctagtgcattgggttacttacattccatcaaaatagtatacagagacttgaaaccagaaaatattcttgtggattcagtaggacatgttgtcttaacagattttgggctttgtaaagaaggaattgctatttctgacaccactaccacattttgtgggacaccagagtatcttgcacctgaagtaattagaaaacagccctatgacaataccgtagattggtggtgccttggggctgttctgtatgaaatgctgtatggattgcctcctttttattgccgagatgttgctgaaatgtatgacaatatccttcacaaacccctaagtttgaggccaggagtgagtcttagagcctggtccattctggaagaactcctagaaaaagacaggcaaaatcgacttggtgccaaggaagactttcttgaaattcagaatcatcctttttttgaatcactcagctgggctgaccttgtacaaaagaagattccaccaccatttaatcctaatgtggctggaccagatgatatcagaaactttgacacagcatttacagaagaaacagttccatattctgtgtgtgtatcttctgactattctatagtgaatgccagtgtattggaggcagatgatgcattcgttggtttctcttatgcacctccttcagaagacttatttttgtgagcagttgccatacagaaaccattgagcaaaataagtctatagatgggactgaaacttctatttgtgtgaatatattcaaatatgtataactagtgcctcatttttatatgtaatgatgaaaactatgaaaaaatgtattttcttctatgtgcaagaaaaatagggcatttcaaagagctgttttgattaaaatttatattcttgtttaataagcttatttttaaacaatttaaaagctattattcttagcattaacctatttttaaagaaacctttttgctattgactgttttttccctctaagtttacactaacatctacccaagatagactgttttttaacagtcaatttcagttcagctaacatatattaatacctttgtaactctttgctatggcttttgttatcacaccaaaactatgcaattggtacatggttgtttaagaagaaaccgtatttttccatgataaatcactgtttgaaatatttggtcatggtatgatcgaaatgtaaaagcataattaacacattggctgctagttaacaattggaataactttattctgcagatcatttaagaagtaacaggccgggcgcgggttgtcacgcctgtaatcccagcactttgggaggctgaggcgggcagatcacctgaggtcaggagttggagaccagcctgaccaacatggacaaaccccgtctctactaaaaatacaaaattggcagggtgtggtgcacatgcctataatcccagctacttgggaggctaaggcaggagaatcgcttgaacccgggaggcggaggttgcagtgagccgagatcgcaccattgcactcctgcctgggcaacaagagtgaaactccatctccaaaaaaaaaaaggaaaaagtaacaaaaggaaattatttgtttttgaaataccagttcaactttgtggattatttttcctctgaaggaaaagaaaaggcttaatggttaggatttttaagtattcccaaagatctgaagggtaataaaatgtactggattttttaaggtggtaccaaaaatgaatgtctgtcatatatttatattacaaatacattatatttatgttctattcatcttttgaatgtttagtatgctattaagtcattctgaatctttgtatttgcttttgcaaataggtatttcaaagctcttttcctaactggttaagtaaaataaaaaattgagctttctagaatatttgcctaattgggaattaaaaagtaaaataataggccaggcatggtggctcatgcctataagcaccctgggaggccgaggcaggcagattatttgagctcaggagtttgagaccatcctgggcaacatggcgaaaccctatctctacaaaaaatacaaaaattagccagacatggtggcacatgcctttagtcccagctactcaggaggctgaagttggaggatggcttgagcccacgagatggaagttgcagtgaactgaaattgtgccactgcacttttcagcctgggtgccacagcgagaccctagttaagagaaaaaaaaagtaaaaacaaattgtgggtcaaagtaaatgtatacagttttattacaatgtaacaaaagttgaaaatcgggcccggaatttatccaatttcagatatatcttagaaaaagcacaggaaacagaccttaaaattgtaacctaccaactaacttacatgcttataaaagtaaaggagaataactggccgggcacggtggctcatgcctataaaattccagcactttgggaggccaaggcaggaggatcacttgagcctatgagttcaagaccagcctaggcaatgtagtgacacctcatttctatttattttaaaaaaaagagagagtaactacagaagaactttaaaaaataaaaaataagcttaccttggattcttggcttagagtagaggttttttttaagttatggaggaaacatttttgtaaaagtttaatgacccactttagatgctccaagaacaagcatcccttccatgtatgtcttgagaaagaaatcacagaagcatttctcaccaatactctttggcttaaaatgttcagcagaattgggcagtgggggtgacttttcttatattaataatatttacatccaatacactgaatcttcctttagaggtaagactttaatatctatactgtaaatatttggtttatttggcactactgtaagttttgtttttcacaaagctcttattatgaagcaaaataaaaattctagtttcttgtatgattttttgtactcattcattcctgttaagctgccaaaaattaaagtgcaatattgtatatttttaagaacaaatttaaaatagaattttgatgtttctcagatcacaagaaatacaaatctatatagtataataaaatcagcaaaagatcaaaaaaaaaaa

SEQ ID NO: 4 - protein, 431 aa, Homo sapiens [sequence omitted]

SEQ ID NO: 5 - protein, 427 aa, Homo sapiens
MQGLLTSGRKPSGGGRCTGRGGWRGQWCLKPWMGGADPPTPTLSCLLLPVPPELPDHCYRMNSSPAGTPSPQPSRANGNINLGPSANPNAQPTDFDFLKVIGKGNYGKVLLAKRKSDGAFYAVKVLQKKSILKKKEQSHIMAERSVLLKNVRHPFLVGLRYSFQTPEKLYFVLDYVNGGELFFHLQRERRFLEPRARFYAAEVASAIGYLHSLNIIYRDLKPENILLDCQGHVVLTDFGLCKEGVEPEDTTSTFCGTPEYLAPEVLRKEPYDRAVDWWCLGAVLYEMLHGLPPFYSQDVSQMYENILHQPLQIPGGRTVAACDLLQSLLHKDQRQRLGSKADFLEIKNHVFFSPINWDDLYHKRLTPPFNPNVTGPADLKHFDPEFTQEAVSKSIGCTPDTVASSSGASSAFLGFSYAPEDDDILDC

SEQ ID NO: 6 - protein, 496 aa, Homo sapiens
MQRDHTMDYKESCPSVSIPSSDEHREKKKRFTVYKVLVSVGRSEWFVFRRYAEVDKLYNTLKKQFPAMALKIPAKRIFGDNFDPDFIKQRRAGLNEFIQNLVRYPELYNHPDVRAFLQMDSPKHQSGPSEDEDERSSQKLHSTSQNINLGPSGNPHAKPTDFDFLKVIGKGSFGKVLLAKRKLDGKVYAVKVLQKKIVLNRKEQKHIMAERNVLLKNVKHPFLVGLHYSFQTTEKLYFVLDFVNGGELFFHLQRERSFPEHRARFYAAEIASALGYLHSIKIVYRDLKPENILVDSVGHVVLTDFGLCKEGIAISDTTTTFCGTPEYLAPEVIRKQPYDNTVDWWCLGAVLYEMLYGLPPFYCRDVAEMYDNILHKPLSLRPGVSLRAWSILEELLEKDRQNRLGAKEDFLEIQNHPFFESLSWADLVQKKIPPPFNPNVAGPDDIRNFDTAFTEETVPYSVCVSSDYSIVNASVLEADDAFVGFSYAPPSEDLFL

SEQ ID NO: 7 - DNA, 846 bp, Homo sapiens
tttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggccc

SEQ ID NO: 8 - DNA, 780 bp, Homo sapiens
gacttcgacttcctcaaagtcatcggcaaagggaactacgggaaggtcctactggccaagcgcaagtctgatggggcgttctatgcagtgaaggtactacagaaaaagtccatcttaaagaagaaagagcagagccacatcatggcagagcgcagtgtgcttctgaagaacgtgcggcaccccttcctcgtgggcctgcgctactccttccagacacctgagaagctctacttcgtgctcgactatgtcaacgggggagagctcttcttccacctgcagcgggagcgccggttcctggagccccgggccaggttctacgctgctgaggtggccagcgccattggctacctgcactccctcaacatcatttacagggatctgaaaccagagaacattctcttggactgccagggacacgtggtgctgacggattttggcctctgcaaggaaggtgtagagcctgaagacaccacatccacattctgtggtacccctgagtacttggcacctgaagtgcttcggaaagagccttatgatcgagcagtggactggtggtgcttgggggcagtcctctacgagatgctccatggcctgccgcccttctacagccaagatgtatcccagatgtatgagaacattctgcaccagccgctacagatccccggaggccggacagtggccgcctgtgacctcctgcaaagccttctccacaaggaccagaggcagcggctgggctccaaagcagactttcttgagattaagaaccatgtattcttcagc

SEQ ID NO: 9 - DNA, 780 bp, Homo sapiens
actttgatttcttaaaagttattggaaaaggcagctttggcaaggttcttcttgcaaaacggaaactggatggaaaattttatgctgtcaaagtgttacagaaaaaaatagttctcaacagaaaagagcaaaaacatattatggctgaacgtaatgtgctcttgaaaaatgtgaaacatccgtttttggttggattgcattattccttccaaacaactgaaaagctttattttgttctggattttgttaatggaggggagctttttttccacttacaaagagaacggtcctttcctgagcacagagctaggttttacgctgctgaaattgctagtgcattgggttacttacattccatcaaaatagtatacagagacttgaaaccagaaaatattcttttggattcagtaggacatgttgtcttaacagattttgggctttgtaaagaaggaattgctatttctgacaccactaccacattttgtgggacaccagagtatcttgcacctgaagtaattagaaaacagccctatgacaatactgtagattggtggtgccttggggctgttctgtatgaaatgctgtatggattgcctcctttttattgccgagatgttgctgaaatgtatgacaatatccttcacaaacccctaagtttgaggccaggagtgagtcttacagcctggtccattctggaagaactcctagaaaaagacaggcaaaatcgacttggtgccaaggaagactttcttgaaattcagaatcatcctttttttgaat

SEQ ID NO: 10 - protein, 260 aa, Homo sapiens
SDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFF

SEQ ID NO: 11 - protein, 260 aa, Homo sapiens
TDFDFLKVIGKGNYGKVLLAKRKSDGAFYAVKVLQKKSILKKKEQSHIMAERSVLLKNVRHPFLVGLRYSFQTPEKLYFVLDYVNGGELFFHLQRERRFLEPRARFYAAEVASAIGYLHSLNIIYRDLKPENILLDCQGHVVLTDFGLCKEGVEPEDTTSTFCGTPEYLAPEVLRKEPYDRAVDWWCLGAVLYEMLHGLPPFYSQDVSQMYENILHQPLQIPGGRTVAACDLLQSLLHKDQRQRLGSKADFLEIKNHVFF

SEQ ID NO: 12 - protein, 260 aa, Homo sapiens
TDFDFLKVIGKGSFGKVLLAKRKLDGKVYAVKVLQKKIVLNRKEQKHIMAERNVLLKNVKHPFLVGLHYSFQTTEKLYFVLDFVNGGELFFHLQRERSFPEHRARFYAAEIASALGYLHSIKIVYRDLKPENILVDSVGHVVLTDFGLCKEGIAISDTTTTFCGTPEYLAPEVIRKQPYDNTVDWWCLGAVLYEMLYGLPPFYCRDVAEMYDNILHKPLSLRPGVSLRAWSILEELLEKDRQNRLGAKEDFLEIQNHPFF

SEQ ID NO: 13 - DNA, 680 bp, Mus musculus
ggcacatcgtcctcactgactttgggctctgcaaagagaatattgagcataacgggacaacatctaccttctgtggcacgcctgagtatctggctcctgaggtcctccataaacagccgtatgaccggacggtggactggtggtgtcttggggctgtcctgtatgagatgctctacggcctgcccccgttttatagccggaacacggctgagatgtacgacaatattctgaacaagcctctccagttgaaaccaaatattacaaactcggcaaggcacctcctggaaggcctcctgcagaaggaccggaccaagaggctgggtgccaaggatgactttatggagattaagagtcatattttcttctctttaattaactgggatgatctcatcaataagaagattacacccccatttaacccaaatgtgagtgggcccagtgaccttcggcacttcgatcccgagtttaccgaggagccggtccccagctccatcggcaggtcccctgacagcatccttgtcacggccagtgtgaaggaagcagcagaagccttcctcggcttctcctatgcacctcctgtggattccttcctctgagtgctcccgggatggttctgaaggacttcctcagcgtttcctaaagtgttttcgttagcctttggtggagttgccagctgacag